(19) United States
(12) Patent Application Publication
(10) Pub. No.: US 2026/0060219 A1
(43) Pub. Date: Mar. 5, 2026

(54) Method for Estimating Amount of Red Muscle, Method for Farming Marine Fish, and Identified Product of Marine Fish

(71) Applicant: Nissui Corporation, Tokyo (JP)

(72) Inventors: Risa Fujii, Saiki-Shi (JP); Takashi Ohta, Saiki-shi (JP); Kagayaki Morishima, Saiki-shi (JP)

(73) Assignee: Nissui Corporation, Tokyo (JP)

(21) Appl. No.: 19/384,091

(22) Filed: Nov. 10, 2025

Related U.S. Application Data
(63) Continuation of application No. PCT/JP2024/014128, filed on Apr. 5, 2024.

(30) Foreign Application Priority Data
May 12, 2023 (JP) 2023-079231

(51) Int. Cl. A01K 61/95; G06T 7/00

(52) U.S. Cl. CPC A01K 61/95; G06T 7/0012; G06T 2207/10132; G06T 2207/30004; G06T 2207/30128

(57) ABSTRACT

A method for estimating the amount of marine fish red muscle which includes acquiring information on the fish's red muscle by operating a non-destructive inspection unit; and estimating the amount of the red muscle included in the fish body based on the acquired information by using a calculation unit. A method for farming a marine fish which includes determining an estimated value of an amount of a red muscle contained in a marine fish body, and adjusting a farming condition for the marine fish based on the estimated value.

### TECHNICAL FIELD

The present disclosure relates to a method for estimating the amount of a red muscle, a method for estimating the amount of a pyloric caeca, a method for producing a processed product of a marine fish, a method for farming a marine fish, an analysis device, an identified product of a marine fish, and a processed product group of a marine fish.

### BACKGROUND ART

In a case where a marine fish is processed into a fillet, a block, sliced raw fish, and the like and distributed, the appearance of the processed product greatly affects the willingness to buy of consumers. For example, a red muscle contained in the body of a marine fish changes in color tone faster than a white muscle. When a processed product of a marine fish contains a red muscle with a changed color tone, the commodity value is reduced. Thus, Patent Document 1 proposes a technique for maintaining the color tone of a red muscle of raw fish. Patent Document 2 proposes a technique for providing a farmed fish in which fading of the red color tone of a red muscle and/or a white muscle after processing is delayed. A marine fish is also known to have a pyloric caeca, which is an organ protruding from the boundary portion between the stomach and the intestine and which secretes digestive enzymes. The pyloric caeca is usually uneaten and often discarded.

### CITATION LIST

### Patent Documents

Patent Document 1: JP2009-232864 A
Patent Document 2: JP2018-29616 A

### SUMMARY OF INVENTION

### Technical Problem

Most of fish meat is a myotomal muscle. The myotomal muscle includes a white muscle and a red muscle. The red muscle differs from the white muscle not only in color tone and taste but also in its change with time. Thus, fish palatability varies depending on the proportions of the white muscle and the red muscle contained in fish meat. However, the proportions of the white muscle and the red muscle can actually be grasped by visual observation only after the red muscle is exposed by processing a fish body into a fillet, a loin, a block, or the like, and it is difficult to artificially adjust the proportions. Similarly, the amount of the pyloric caeca can be determined by visual observation after the pyloric caeca is exposed by processing the fish body.

The present disclosure provides an estimation method and an analysis device capable of smoothly estimating the amount of red muscle of a marine fish without exposing the red muscle. Provided are an estimation method and an analysis device capable of smoothly estimating the amount of the pyloric caeca of a marine fish without exposing the pyloric caeca. Also provided is a farming method capable of efficiently and flexibly providing a marine fish that meet consumer preferences, thereby ensuring added value. There is provided a method for producing a processed marine fish product is provided, capable of efficiently yielding a processed product of a marine fish having added value flexibly to provide products that meet consumer preferences. There is provided a processed product group of a marine fish having a sufficiently reduced variation in the amount of red muscle or pyloric caeca and added value. A marine fish and a prepared product thereof using the described methods and having added value are also provided.

### Solution to the Problem

One aspect of the present disclosure provides a method for estimating an amount of a red muscle, the method comprising: an information acquisition step of acquiring information on a red fish muscle of a marine fish by non-destructive inspection; and an estimation step of estimating the amount of the red muscle included in the fish body based on the information.

In the estimation method, the amount of the red muscle is estimated based on the information on the red muscle acquired by the non-destructive inspection. Thus, the amount of the red fish muscle can be easily estimated without exposing the red muscle. For example, it is possible to estimate the amount of a red muscle of a marine fish in a living state. When this estimation method is used for, for example, marine fish farming, a fish having added value can be efficiently provided to meet consumer preferences.

One aspect of the present disclosure provides a method for producing a processed product of a marine fish, the method comprising a step of processing a marine fish in which an amount of a red muscle is estimated by the estimation method described herein.

In the above production method, the marine fish in which the amount of a red muscle is estimated by the above estimation method is processed, and thus, it is possible to efficiently provide a processed product of a marine fish having added value flexibly to provide products that meet consumer preferences.

One aspect of the present disclosure provides a method for farming a marine fish, the method comprising using an estimated value of the amount of a red muscle contained in a fish body of a marine fish as an index in any step of a farming process.

In the above farming method, it is possible to selectively obtain a farmed fish with a small amount of a red muscle by selecting breeding based on the estimated value of the amount of the red muscle, for example. This makes it possible to efficiently and consistently provide a marine fish having added value.

One aspect of the present disclosure provides an analysis device comprising: a non-destructive inspection unit that acquires information on a red muscle of a fish body of a marine fish by non-destructive inspection; and a red muscle estimation unit that estimates an amount of the red muscle of the fish body based on the information.

The analysis device estimates the amount of the red muscle based on the information on the red muscle acquired by the non-destructive inspection. This makes it possible to smoothly estimate the amount of the red muscle of the marine fish without exposing the red muscle. For example, it is possible to estimate the amount of a red muscle of a marine fish in a living state. When this analysis device is used for, for example, farming of a marine fish, a marine fish having added value can be efficiently provided to meet consumer preferences.

One aspect of the present disclosure provides an identified product of a marine fish comprising: a fish body or a processed product of a marine fish; and an identification portion including information indicating that an amount of a red muscle contained in the fish body or the processed product has been estimated by non-destructive inspection.

The identified product has the identification portion including the information indicating that the amount of the red muscle contained in the fish body or the processed product of the marine fish has been estimated. This makes it possible for a user or an end user to select a fish body based on the information. For example, an identified product with higher red muscle content would have greater market value to consumers.

One aspect of the present disclosure provides a processed product group of a marine fish, comprising a plurality of processed products of a marine fish, wherein a standard deviation of weight ratios of red muscles included in the plurality of processed products is 1.5 wt. % or less. In such a processed marine fish product group, the variation in weight of the red muscle is sufficiently reduced. Thus, the processed product group has high quality, reliability and added value.

One aspect of the present disclosure provides a processed product group of a marine fish obtained by processing a first marine fish that is a part of a marine fish, the first marine fish being sorted in the step of selecting a marine fish in the above-described farming method. In such a processed product group of a marine fish, the variation in red muscle amount is sufficiently reduced. When a processed product group of a marine fish is rated as high in quality using the described method, the method has been shown to produce consistent and reliable results. Such reliable methods of non-destructive fish analysis meet the market's need for consistently determining the amount of red muscle tissue, which, in turn, can provide added value for fish with known red muscle tissue.

One aspect of the present disclosure provides a method for estimating the amount of a pyloric caeca, the method comprising: an information acquisition step of acquiring information on a pyloric caeca in a fish body of a marine fish by non-destructive inspection; and an estimation step of estimating an amount of the pyloric caeca contained in the fish body based on the information.

In the estimation method, the amount of the pyloric caeca is estimated based on the information on the pyloric caeca acquired by the non-destructive inspection. This makes it possible to efficiently estimate the amount of the pyloric caeca of a marine fish without exposing the pyloric caeca. For example, it is possible to estimate the amount of a pyloric caeca of a marine fish in a living state. When this estimation method is used for, for example, farming of a marine fish, it is possible to efficiently provide a marine fish having added value to provide products that meet consumer preferences.

One aspect of the present disclosure provides a method for producing a processed product of a marine fish, the method comprising a step of processing a marine fish in which the amount of a pyloric caeca has been estimated by the estimation method described herein and above.

In the above production method, the marine fish in which the amount of the pyloric caeca has been estimated by the disclosed estimation method is processed, and thus, it is possible to efficiently provide a processed product of a marine fish having a added value flexibly to provide products that meet consumer preferences.

One aspect of the present disclosure provides a method for farming a marine fish, the method comprising using an estimated value of the amount of a pyloric caeca contained in a fish body of a marine fish as an index in any step of a farming process. As the relationship between the non-destructive inspection data and the actual amount may vary depending on the fish species, the estimation can be performed using statistical data or a model prepared for each species to improve accuracy.

In the above farming method, for example, it is possible to selectively obtain a farmed fish having a small amount of a pyloric caeca by selecting breeding based on the estimated value of the amount of the pyloric caeca. This makes it possible to efficiently provide a marine fish having added value.

One aspect of the present disclosure provides an analysis device comprising: a non-destructive inspection unit that acquires information on a pyloric caeca in a fish body of a marine fish by non-destructive inspection of the fish; and a pyloric caeca estimation unit that estimates the amount of the pyloric caeca included in the fish body based on the information. By non-destructive fish inspection is meant to analyze either the fish while alive or prior to the fish being processed for product manufacture.

The analysis device estimates the amount of the pyloric caeca based on the information on the pyloric caeca acquired by the non-destructive inspection. This makes it possible to efficiently estimate the amount of a pyloric caeca of a marine fish without exposing the pyloric caeca. For example, it is possible to estimate the amount of a pyloric caeca of a marine fish in a living state. When this analysis device is used for, for example, farming of a marine fish, it is possible to efficiently provide a marine fish having added value to provide products that meet consumer preferences.

One aspect of the present disclosure provides an identified product of a marine fish, comprising: a fish body or a processed product of a marine fish; and an identification portion including information that the amount of the pyloric caeca contained in the fish body or the amount of the pyloric caeca in the processed product has been estimated by non-destructive inspection.

The identified product has the identification portion including the information indicating that the amount of the pyloric caeca contained in the whole fish body or the processed product of a portion of the whole fish has been estimated. This makes it possible for a user or an end user to select a fish body based on the information. Such an identified product has added value.

One aspect of the present disclosure provides a processed product group of a marine fish, comprising a plurality of processed products of a marine fish, wherein a standard deviation of weight ratios of pyloric caeca contained in the plurality of processed products is 10.0 wt. % or less. In such a processed product group of a marine fish, the variation in weight of the pyloric caeca is sufficiently reduced. Accordingly, the processed product group has high quality, reliability and added value.

One aspect of the present disclosure provides a processed product group of a marine fish obtained by processing a first marine fish that is a part of a marine fish, the first marine fish being sorted in the step of selecting a marine fish in the above-described farming method. In such a processed product group of a marine fish, the variation in the amount of the pyloric caeca is sufficiently reduced. Accordingly, quality reliability is high, and the yield can be increased, and thus the processed product group has added value.

### Advantageous Effects of Invention

The present disclosure can provide an estimation method and an analysis device capable of smoothly estimating the amount of red muscle in a marine fish while the fish is alive, or without exposing the fish's red muscle. It is possible to provide an estimation method and an analysis device capable of smoothly estimating the amount of a pyloric caeca of a marine fish without exposing the pyloric caeca. It is possible to provide a method for farming a marine fish, the method being capable of efficiently and flexibly providing a marine fish that meet consumer preferences, thereby ensuring added value. It is possible to provide a method for producing a processed product of a marine fish, the method being capable of efficiently providing a processed product of a marine fish having added value flexibly to provide products that meet consumer preferences. The described method provides a means to analyze a processed product group of a marine fish that can sufficiently and reliably determine the amount of a red muscle which can thus increase the group's market value based on the reliability of the analysis. It is possible to provide a processed product group of a marine fish having sufficiently reduced variation in the amount of a pyloric caeca and having added value. It is possible to provide an identified product of a marine fish having added value.

### BRIEF DESCRIPTION OF DRAWING

FIG. 1 is a diagram illustrating an example of an analysis device.

FIG. 2 is a diagram schematically illustrating a measurement site of a fish body to which a probe is applied.

FIG. 3 is a diagram illustrating an example of an identified product of a marine fish.

FIG. 4A is a graph showing a relationship between a cross-sectional area of a red muscle and a weight of the red muscle in a pelvic fin beginning portion. FIG. 4B is a graph showing a relationship between a cross-sectional area of a red muscle and a weight of the red muscle in a second dorsal fin beginning portion. FIG. 4C is a graph showing a relationship between a cross-sectional area of a red muscle and a weight of the red muscle in an anus.

FIG. 5A provides a transverse section echo image and a photograph of a cut surface in the vicinity of a dorsal fin root, and FIG. 5B provides a transverse section echo image and a photograph of a cut surface in the vicinity of an anus.

FIG. 6A and FIG. 6B are graphs showing a relationship between a cross-sectional area of a red muscle determined from the transverse section echo image and the weight of the red muscle actually measured respectively.

### DESCRIPTION OF EMBODIMENTS

Hereinafter, embodiments of the present disclosure will be described, sometimes with reference to the drawings. However, the following embodiments are examples for describing the present disclosure, and are not intended to limit the present disclosure to the following contents. In the description, the same elements or elements having the same function are denoted by the same reference numerals, and redundant descriptions may be omitted. Unless otherwise specified, positional relationships such as up, down, left, and right are based on positional relationships when directions of the reference numerals shown in the drawings are used as references. The dimensional ratios of the respective elements are not limited to the ratios shown in the drawings. In the present embodiments, a numerical range indicated by “to” includes numerical values indicated as the upper limit and the lower limit. In addition, numerical ranges in which the upper limit and the lower limit of a numerical range are replaced with values of Examples are also included in the present disclosure. In a case where a plurality of numerical ranges are exemplified in a stepwise manner, a numerical range in which an upper limit value or a lower limit value in a first numerical range is replaced with an upper limit value or a lower limit value in a second numerical range narrower than the first numerical range is also included in the present disclosure. Substances and materials exemplified in plural numbers may be used alone or in combination of two or more kinds arbitrarily selected.

### Method for Estimating the Amount of Red Muscle in a Marine Fish

A method for estimating the amount of a red muscle according to an embodiment may include: an information acquisition step of acquiring information (first information) on a red muscle in a fish body of a marine fish by non-destructive inspection; and an estimation step of estimating the amount of the red muscle contained in the fish body based on the first information.

In the present specification, fish species of the marine fish are not particularly limited. Examples of fish species in which a color tone of the red muscle has a large influence on a commodity value include a marine fish belonging to the order Perciformes. For example, a marine fish belonging to the family Scombridae or the family Carangidae is exemplified as a marine fish belonging to the order Perciformes, in which the color tone of the red muscle has a significant influence on the market value of the fish products obtained therefrom. Examples of fish species in which the amount of the red muscle has a particularly large impact on the commodity value include marine fish belonging to the genus Thunnus, the genus Katsuwonus, the genus Euthynnus, and the genus Auxis of the family Scombridae; and the genus Seriola, the genus Trachurus, the genus Decapterus, the genus Pseudocaranx, the genus Megalaspis, and the genus Caranx of the family Carangidae. The marine fish may include, as a fish suitable for farming, at least one selected from the group consisting of yellowtail (Seriola quinqueradiata also known as Japanese amberjack), great amberjack (Seriola dumerili, also known as greater amberjack, allied kingfish, and greater yellowtail), yellowtail amberjack (Seriola lalandi), white trevally (Pseudocaranx dentex also known as striped jack), Pacific bluefin tuna (Thunnus orientalis), Atlantic bluefin tuna (Thunnus thynnus), and southern bluefin tuna (Thunnus maccoyii). In a case of a farmed fish, the amount of a red muscle can be easily adjusted in a rearing environment by measuring the amount of the red muscle in a non-destructive manner using the method and device described herein.

The non-destructive inspection in the present specification is a method of inspecting a fish body without destroying the fish body, and can be performed using a commercially available non-destructive inspection device. As the non-destructive inspection means or device, at least one selected from the group consisting of ultrasonic inspection, MRI inspection, and CT inspection. Among these, from the viewpoint of workability and convenience in a farm, ultrasonic inspection may be used.

The fish body may or may not be alive. When a living fish body is used, the estimation result of the red muscle can be used for follow-up observation of a farmed fish, selection of breeding, and the like. This makes it possible to preferentially farm or breed a fish body with a small amount of a red muscle, for example.

In acquiring the first information, a living fish body may be immobilized using anesthesia. This makes it possible to smoothly acquire the information on the red muscle. In addition, the amount of the red muscle can be estimated with higher accuracy. The anesthesia method is not particularly limited as described below. However, it is not essential to apply anesthesia, and for example, information on the red muscle of a fish body that is migratory in a water tank may be acquired using a non-destructive inspection device provided inside or around the water tank.

The first information may be acquired by observing the red muscle from an image of a fish body of a marine fish, or may be acquired by estimating an amount of a white muscle from the image and subtracting the estimated amount of the white muscle from the entire muscles.

The first information may be a three-dimensional image or a two-dimensional image. The two-dimensional image is preferable because it can be acquired more easily and quickly than the three-dimensional image. The first information may include a cross-sectional image of the fish body, and may include a cross-sectional area of the red muscle determined from the cross-sectional image. The cross-sectional image may include a longitudinal section image of the fish body or may include a transverse section image thereof. In the present specification, a transverse section refers to a cross-section orthogonal to a direction in which a spine extends, and a longitudinal section refers to a cross-section parallel to the direction in which the spine extends. From the viewpoint of the size of a detector, such as a probe used for measurement, the cross-sectional image may be a transverse section image of the fish body. The cross-sectional image may include a transverse section image that measures a fish between the fourth vertebra and the twenty-second vertebra, between the second dorsal fin beginning portion and the vicinity of the anus, or between the second dorsal fin beginning portion and the anal fin beginning portion. This makes it possible to estimate the amount of the red muscle with high accuracy. The cross-sectional image may include a transverse section image between the eleventh vertebra to the seventeenth vertebra of the marine fish. This makes it possible to estimate the amount of the red muscle with higher accuracy. From the viewpoint of estimating the amount of the red muscle with higher accuracy, the cross-sectional image may include a transverse section image passing through the anus of the fish body. That is, the cross-sectional image may include a transverse section image containing at least a portion of the anus of the fish body.

The cross-sectional area of the red muscle included in the first information may be determined from the cross-sectional image using image processing software, or may be determined using a measurement function provided in the non-destructive inspection device. Alternatively, an operator may calculate the length or the cross-sectional area using a cross-sectional image displayed on the monitor or a printed photograph. When the first information includes the cross-sectional area of the red muscle determined from the cross-sectional image, it is possible to estimate the amount of the red muscle with high accuracy.

In the estimation step, the amount of the red muscle is estimated based on the first information. The amount of the red muscle estimated in the estimation step may be the weight of the red muscle or the volume of the red muscle. A relationship (correlation equation) between the cross-sectional area of the red muscle and the amount of the red muscle may vary depending on the weight of the fish body. Accordingly, a weight measurement step of measuring the weight of the fish body may be performed before the information acquisition step or between the information acquisition step and the estimation step. This makes it possible to select a correlation equation depending on the weight of the fish body. Thus, the amount of the red muscle can be estimated with higher accuracy.

Statistical data with or without a machine learning (ML) model may be used to estimate the amount of the red muscle in the estimation step. The statistical data may include, for example, a correlation equation obtained by performing statistical processing of the cross-sectional area of the red muscle and the weight of the red muscle included in the fish body that is actually measured. Such a correlation equation can be obtained by performing statistical processing of the cross-sectional area of the red muscle included in the fish body and the weight of the red muscle that is actually measured. The weight of the red muscle can be measured by, for example, removing vertebrae from a meat mass after fillet processing, and dividing the resulting meat mass into the red muscle and the white muscle. Such a correlation equation may be prepared for each weight of the fish body. The relationship between the first information and the amount of the red muscle may vary depending on fish species. Thus, prediction accuracy can be improved by preparing statistical data for each fish species to be analyzed using the methods described herein. The white muscle may be used for estimating the amount of red muscle tissue, similarly to the red muscle. On the other hand, the weight of the white muscle is different from that of the red muscle, and may vary depending on the degree of processing of the product in terms of how far the head is included during fillet processing and the high possibility that the white muscle is also removed together during trimming of the abdominal bone and the like. Thus, it is preferable to use the red muscle rather than the white muscle for estimating the amount of red muscle tissue.

The machine learning model may be constructed using, for example, the cross-sectional area of the red muscle included in the fish body, and the weight of the fish body and the weight of the red muscle that are actually measured as training data. In this case as well, the training data may be prepared for each fish species for learning. The machine learning model may have an algorithm for increasing prediction accuracy based on the deviation between a predicted value and an actual value of the amount of the red muscle.

In the estimation method, the amount such as the weight and/or the volume of the red muscle is estimated based on the information on the red muscle acquired by the non-destructive inspection. Thus, the amount of a red muscle of a marine fish can be smoothly estimated without exposing the red muscle. For example, it is possible to estimate the amount of a red muscle of a marine fish in a living state. Until now, there has been no method for evaluating a red muscle without first processing a fish, collecting the red fish muscle, and measuring the proportion of red fish muscle. However, with such a method, a marine fish cannot be kept alive. Using the methods described herein, farmed marine fish can be analyzed and grown to more reliably and effectively produce a high red meat content, thereby producing marine fish having a higher market value and meeting consumer demand. These methods will therefore help improve the marine aquaculture of fish more efficiently and economically. This estimation method may be performed using the analysis device described below. Thus, the following description of the analysis device also applies to this estimation method. Moreover, the description of the estimation method described above also applies to the analysis device, a farming method, a method for producing a processed product of a marine fish, an identified product of a marine fish, and a processed product group of a marine fish below.

### Method for Estimating the Amount of Pyloric Caeca

A method for estimating the amount of a pyloric caeca according to an embodiment includes: an information acquisition step of acquiring information (third information) on a pyloric caeca in a fish body of a marine fish by non-destructive inspection; and an estimation step of estimating an amount of the pyloric caeca contained in the fish body based on the third information.

In the present specification, fish species of the marine fish are not particularly limited. Examples of a fish species in which the pyloric caeca has a large influence on a commodity value by a yield include a marine fish belonging to the order Perciformes. For example, a marine fish belonging to the family Scombridae or the family Carangidae is exemplified as the marine fish belonging to the order Perciformes, in which the pyloric caeca has an influence on a commodity value by a yield. Examples of fish species in which the amount of the pyloric caeca has a particularly large influence on the commodity value include marine fish belonging to the genus Thunnus, the genus Katsuwonus, the genus Euthynnus, and the genus Auxis of the family Scombridae, and the genus Seriola, the genus Trachurus, the genus Decapterus, the genus Pseudocaranx, the genus Megalaspis, and the genus Caranx of the family Carangidae. The marine fish may include, as a fish suitable for farming, at least one selected from the group consisting of yellowtail (Seriola quinqueradiata), great amberjack (Seriola dumerili), yellowtail amberjack (Seriola lalandi), white trevally (Pseudocaranx dentex), Pacific bluefin tuna (Thunnus orientalis), Atlantic bluefin tuna (Thunnus thynnus), and southern bluefin tuna (Thunnus maccoyii). In the case of farmed fish, the evaluation may be important because they are often fed formula feed, which can increase the number of pyloric caeca and can be further improved to increase their number and size further. A higher number of pyloric caeca increases a fish's digestive surface area and nutrient absorption, allowing for faster fish growth.

The non-destructive inspection in the present specification is a method of inspecting a fish body without destroying the fish's body, and can be performed using a commercially available non-destructive inspection device. As the non-destructive inspection, at least one selected from the group consisting of ultrasonic inspection, MRI inspection, and CT inspection may be used. Among these, from the viewpoint of workability and convenience in a farm, ultrasonic inspection may be used.

The fish may or may not be alive. When a living fish is used, the estimation result of the pyloric caeca can be used for follow-up observation of a farmed fish, selection of breeding, improvements to fish feed, and the like. This makes it possible to preferentially farm or breed a fish body with a small amount of a pyloric caeca, for example.

To obtain the third piece of information, a living fish may be immobilized using anesthesia. This makes it possible to easily acquire the information on the pyloric caeca. In addition, the amount of the pyloric caeca can be estimated with higher accuracy. The anesthesia method is not particularly limited as described below. However, it is not essential to apply anesthesia, and for example, information on the pyloric caeca of a fish in the water tank may be acquired using a non-destructive inspection device provided inside or around the water tank. In obtaining the third piece of information, the position of a pyloric caeca cross-section at which the pyloric caeca weight can be appropriately evaluated may be specified in advance. When the position is specified, the pyloric caeca weight can be estimated more accurately from the cross-sectional area.

The third piece of information may be a three-dimensional image or a two-dimensional image. The third piece of information may include a cross-sectional image of the fish body, and may include a cross-sectional area of the pyloric caeca determined from the cross-sectional image. The cross-sectional image may be a longitudinal section image of the fish body or a transverse section image. In the present specification, a transverse section refers to a cross-section orthogonal to a direction in which a spine extends, and a longitudinal section refers to a cross-section parallel to the direction in which the spine extends. From the viewpoint of the size of a detector, such as a probe used for measurement using the described methods, the cross-sectional image may be a transverse section image of the fish body. The cross-sectional image may include a transverse section image spanning the second vertebra to the eleventh vertebra of the marine fish. This makes it possible to estimate the amount of the pyloric caeca with high accuracy. The cross-sectional image may include a transverse section image between the fourth vertebra and the ninth vertebra of the marine fish, or in the vicinity of a pelvic fin root. This makes it possible to estimate the amount of the pyloric caeca with higher accuracy. From the viewpoint of estimating the amount of the pyloric caeca with higher accuracy, the cross-sectional image may include a transverse section image at the pelvic fin root of the fish body. That is, the cross-sectional image may include a transverse section image containing at least a portion of the pelvic fin root of the fish body.

The cross-sectional area of the pyloric caeca included in the third piece of information may be determined from the cross-sectional image using image processing software, or may be determined using a measurement function provided in the non-destructive inspection device. Alternatively, an operator may calculate the length or the cross-sectional area using a cross-sectional image displayed on the monitor or a printed photograph. When the third information includes the cross-sectional area of the pyloric caeca determined from the cross-sectional image, it is possible to estimate the amount of the pyloric caeca with high accuracy.

In the estimation step, the amount of the pyloric caeca is estimated based on the third information. The amount of the pyloric caeca estimated in the estimation step may be the weight of the pyloric caeca or the volume of the pyloric caeca. A relationship (correlation equation) between the cross-sectional area of the pyloric caeca and the amount of the pyloric caeca may vary depending on the weight of the fish. Accordingly, a weight measurement step of measuring the weight of the fish body may be performed before the information acquisition step or between the information acquisition step and the estimation step. This makes it possible to select a correlation equation depending on the weight of the fish body. Thus, the amount of the pyloric caeca can be estimated with higher accuracy.

Statistical data or a machine learning model may be used to estimate the amount of the pyloric caeca in the estimation step. The statistical data may include, for example, a correlation equation obtained by performing statistical processing of the cross-sectional area of the pyloric caeca and the weight of the pyloric caeca contained in the fish body that is actually measured. Such a correlation equation can be obtained by performing statistical processing of the cross-sectional area of the pyloric caeca contained in the fish body and the weight of the pyloric caeca that is actually measured. Such a correlation equation also may be prepared for each weight of the fish body. The relationship between the third piece of information and the amount of the pyloric caeca may vary depending on fish species. Thus, prediction accuracy can be improved by preparing statistical data for each fish species.

A machine learning model may be constructed using, for example, data obtained from the cross-sectional area of a pyloric caeca contained in a fish body, and the weight of the fish body and the weight of the pyloric caeca that are actually measured, as training data. In this case as well, the training data may be prepared for each fish species for learning. The machine learning model may have an algorithm for increasing the prediction accuracy based on the deviation between a predicted value and an actual value of the amount of the pyloric caeca.

In one estimation method, the amount, such as the weight and/or the volume of the pyloric caeca, is estimated based on the information of the pyloric caeca acquired by the non-destructive inspection. Thus, the pyloric caeca amount of a marine fish can be smoothly estimated without exposing the pyloric caeca. For example, it is possible to estimate the amount of a pyloric caeca of a marine fish in a living state. Until now, there has been no method available to evaluate a fish's pyloric caeca without harvesting and processing the fish to collect and measure the fish's pyloric caeca directly. However, with such a method, a marine fish cannot be kept alive. Accordingly, when the above estimation method is applied to, for example, farming of a marine fish, a marine fish having added value can be more effectively and efficiently grown to provide products that meet consumer preferences. This estimation method may be performed using an analysis device to be described below. Thus, the following description of the analysis device also applies to this estimation method. Moreover, the description of the estimation method described above also applies to the analysis device, a farming method, a method for producing a processed product of a marine fish, an identified product of a marine fish, and a processed product group of a marine fish below.

### Analysis Device for Red Muscle

An analysis device for a red muscle according to an embodiment includes: a non-destructive inspection unit or device that acquires first information on a red muscle in a fish body of a marine fish by non-destructive inspection; and a red muscle estimation unit that estimates an amount of the red muscle contained in the fish body based on the first information. The analysis device can analyze a fish without destroying the fish body during inspection and even while the fish remains alive. The non-destructive inspection unit may include at least one selected from the group consisting of an ultrasonic inspection unit, an MRI inspection unit, and a CT inspection unit. As each inspection unit, a commercially available inspection unit may be used. The non-destructive inspection unit may include an ultrasonic inspection unit from the viewpoint of workability and convenience on a farm. The ultrasonic inspection unit is inexpensive and small in size, and thus has the advantage of being easily brought into a farm and used. However, the non-destructive inspection unit is not limited to the ultrasonic inspection unit, and may alternatively be an MRI inspection unit or a CT inspection unit. These have the advantage of being excellent in capturing data on the entire fish body and acquiring a clearer image. The non-destructive inspection units may further include a calculation unit that calculates a cross-sectional area of the red muscle from the cross-sectional image of the fish body and outputs the first piece of information, including the cross-sectional area. The red muscle estimation unit may include a calculation unit to which the first information is input, the calculation unit performing calculations using statistical data or a machine learning model, and outputting an estimated amount of red muscle.

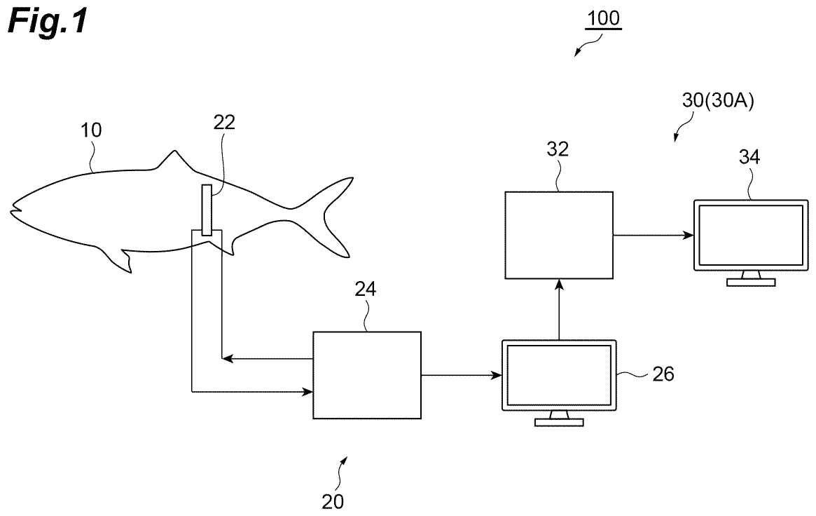

FIG. 1 is a diagram illustrating an example of an analysis device for a red muscle, including an ultrasonic inspection unit. An analysis device 100 includes a non-destructive inspection unit 20 and a red muscle estimation unit 30. The non-destructive inspection unit 20 includes a probe 22 having an ultrasonic transducer that transmits an ultrasonic wave and detects a reflected wave (echo) reflected from a tissue of a fish body 10, a control unit 24 that exchanges signals with the probe 22 and outputs an image signal to be displayed on a first display unit 26, and the first display unit 26 on which a cross-sectional image (echo image) is displayed based on the image signal.

The probe 22 is applied to a measurement site of the fish body 10 to detect a reflected wave. An operator may calculate a length or a cross-sectional area of the cross-section of the red muscle using the cross-sectional image of the fish body 10 displayed on the first display unit 26 or a printed photograph. Alternatively, the control unit 24 may calculate the length or the cross-sectional area of the cross-section of the red muscle and display the calculated length or the calculated cross-sectional area on the first display unit 26. The control unit 24 may include, for example, a transmission unit that generates an electric pulse signal to be applied to the ultrasonic transducer, a reception unit that receives a reflection signal from the probe 22, a converter that converts the reflection signal received by the reception unit into an image signal, a calculation unit that calculates a cross-sectional area of the red muscle from a cross-sectional image, and the like. The control unit 24 and the first display unit 26 may be configured as separate pieces of hardware or may be integrated. The non-destructive inspection unit 20 may be composed of a commercially available ultrasonic inspection device. The first display unit 26 may be composed of a general monitor.

The red muscle estimation unit 30 includes a calculation unit 32 that calculates an estimated value of the amount of the red muscle based on the first information displayed on the first display unit 26, and a second display unit 34 on which the estimated value calculated by the calculation unit 32 is displayed. The calculation unit 32 receives the first piece of information, performs a calculation using the statistical data or a machine learning model, and outputs an estimated value of the amount of the red muscle. On the second display unit 34, the estimated value of the amount of the red muscle is displayed. The calculation unit 32 of the red muscle estimation unit 30 may be composed of a general computer, and the second display unit 34 may be composed of a general monitor.

Although the control unit 24 and the calculation unit 32 are illustrated as separate components in FIG. 1, these components may be configured as one piece of hardware or may be configured as separate pieces of hardware. The first display unit 26 and the second display unit 34 may also be separate monitors or may be configured as a single monitor. In the latter case, the display of the cross-sectional image and the display of the estimated value of the amount of the red muscle may be switched, or the cross-sectional image and the estimated value of the amount of the red muscle may be displayed at the same time.

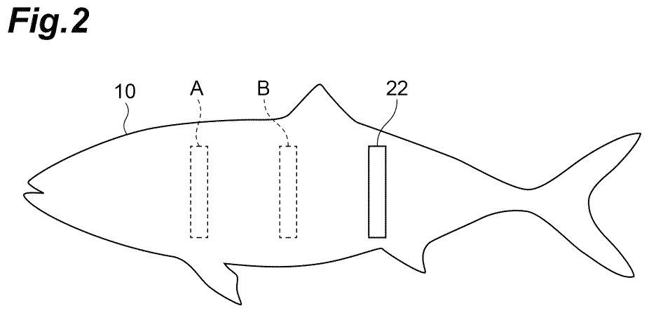

In FIG. 1, the probe 22 is applied to the vicinity of the anus of the fish body 10 to detect a reflected wave. The transverse section image of the vicinity of the anus includes a part of a transverse section obtained by cutting the fish body 10 to pass through the anus or a position shifted from the anus by 0 to 5% of the entire length of the fish body 10 along the length direction of the fish body 10. The measurement site to which the probe 22 is applied is not limited to the vicinity of the anus. As illustrated in FIG. 2, the probe 22 may be moved to the front of the fish body 10 and placed at a position A to obtain a cross-sectional image of a pelvic fin beginning portion of the fish body 10. The probe 22 may be placed at a position B to obtain a cross-sectional image of the second dorsal fin beginning portion.

Although the ultrasonic inspection unit is illustrated in the example of FIG. 1, the non-destructive inspection unit 20 may include an MRI inspection unit or a CT inspection unit instead of the ultrasonic inspection unit. For example, when the non-destructive inspection unit 20 includes an MRI inspection unit, the non-destructive inspection unit 20 may include a superconducting magnet that generates a magnetic field, a transmission unit (high-frequency coil) that transmits an electric wave, a gradient magnetic field coil unit that changes the magnetic field, a reception unit (reception coil) that receives an electric wave from the inside of the fish body, and the like. The MRI inspection unit can obtain a cross-sectional image of the fish body by using magnetism, an electromagnetic wave, and movement of a hydrogen atom. In a case where the non-destructive inspection unit 20 includes a CT inspection unit, the CT inspection unit may include an X-ray tube that irradiates the fish body with X-rays, a detector that detects an X-ray that has passed through the fish body, and a drive unit that rotationally drives the X-ray tube and the detector. As the MRI inspection unit and the CT inspection unit, a commercially available MRI inspection device and a commercially available CT inspection device can be used.

The analysis device 100 estimates the amount of the red muscle based on the first information of the red muscle acquired by the non-destructive inspection. Thus, the amount of a red muscle of a marine fish can be smoothly estimated without exposing the red muscle. For example, it is possible to estimate the amount of a living red marine fish muscle. Anesthesia may be applied to the marine fish prior to the non-destructive inspection. By applying anesthesia, an image can be stably acquired in the non-destructive inspection. When the marine fish is returned to seawater immediately after the non-destructive inspection, the marine fish can be awakened from anesthesia. The anesthesia method is not particularly limited, as long as it can immobilize the fish for a time sufficient to obtain a stable image for non-destructive inspection using any of the described methods. For example, a marine fish may be immobilized by anesthesia with an anesthetic such as 2-phenoxyethanol or eugenol, or by shock with electric shock, a sound, or a vibration. When the analysis device 100 is used for, for example, farming of a marine fish, it is possible to efficiently provide a marine fish having added value to provide products that meet consumer preferences. Accordingly, the description of the analysis device described above also applies to the description of the farming method below

### Analysis Device for Pyloric Caeca

An analysis device for a pyloric caeca according to an embodiment includes: a non-destructive inspection unit that acquires third information on a pyloric caeca in a fish body of a marine fish by non-destructive inspection; and a pyloric caeca estimation unit that estimates an amount of the pyloric caeca contained in the fish body based on the third information. The analysis device is a device capable of analyzing a fish body without destroying the fish body or requiring the fish to be dead during inspection. The non-destructive inspection unit may include at least one selected from the group consisting of an ultrasonic inspection unit, an MRI inspection unit, and a CT inspection unit. An inspection unit can be a commercially available inspection unit. The non-destructive inspection unit may include an ultrasonic inspection unit from the viewpoint of workability and convenience on a farm. The ultrasonic inspection unit is inexpensive and small in size, and thus has the advantage of being easily brought into a farm and easily used. However, the non-destructive inspection unit is not limited to the ultrasonic inspection unit, and may be an MRI inspection unit or a CT inspection unit. These have the advantage of being excellent in capturing the entire fish body and being capable of acquiring a clearer image. The non-destructive inspection unit may include a calculation unit that calculates a cross-sectional area of the pyloric caeca from the cross-sectional image of the fish body and outputs the third piece of information, including the cross-sectional area. The pyloric caeca estimation unit may include a calculation unit to which the third information is input, the calculation unit performing calculation using statistical data or a machine learning model, and outputting an estimated value of the amount of the pyloric caeca.

FIG. 1 is a diagram illustrating an example of an analysis device for a pyloric caeca including an ultrasonic inspection unit. An analysis device 100 includes a non-destructive inspection unit 20 and a pyloric caeca estimation unit 30A. The non-destructive inspection unit 20 includes a probe 22 having an ultrasonic transducer that transmits an ultrasonic wave and detects a reflected wave (echo) reflected from a tissue of a fish body 10, a control unit 24 that exchanges signals with the probe 22 and outputs an image signal to be displayed on a first display unit 26, and the first display unit 26 on which a cross-sectional image (echo image) is displayed based on the image signal.

As illustrated in FIG. 2, the probe 22 is applied to a measurement site of the fish body 10 to detect a reflected wave. An operator may calculate a length or a cross-sectional area of the cross-section of the pyloric caeca using the cross-sectional image of the fish body 10 displayed on the first display unit 26 in FIG. 1 or a printed photograph. Alternatively, the control unit 24 may calculate the length or the cross-sectional area of the cross-section of the pyloric caeca and display the calculated length or the calculated cross-sectional area on the first display unit. The control unit 24 may include, for example, a transmission unit that generates an electric pulse signal to be applied to an ultrasonic transducer, a reception unit that receives a reflection signal from the probe 22, a converter that converts the reflection signal received by the reception unit into an image signal, a calculation unit that calculates the cross-sectional area of the pyloric caeca from a cross-sectional image, and the like. The control unit 24 and the first display unit 26 may be configured as separate pieces of hardware or may be integrated into one unit. The non-destructive inspection unit 20 may be composed of a commercially available ultrasonic inspection device. The first display unit 26 may be composed of a general monitor.

The pyloric caeca estimation unit 30A includes a calculation unit 32 that calculates an estimated value of the amount of the pyloric caeca based on the third information displayed on the first display unit 26, and a second display unit 34 on which the estimated value calculated by the calculation unit 32 is displayed. The calculation unit 32 receives the third piece of information, performs a calculation on the third data point using statistical data or a machine learning model, and outputs an estimated value of the amount of the pyloric caeca. On the second display unit 34, the estimated value of the amount of the pyloric caeca is displayed. The calculation unit 32 of the pyloric caeca estimation unit 30A may be composed of a general computer, and the second display unit 34 may be composed of a general monitor.

Although the control unit 24 and the calculation unit 32 are illustrated as separate components in FIG. 1, these components may be configured as one piece of hardware or may be configured as separate pieces of hardware. The first display unit 26 and the second display unit 34 may also be separate monitors or may be configured as a single monitor. In the latter case, the display of the cross-sectional image and the display of the estimated value of the pyloric caeca amount may be switched, or the cross-sectional image and the estimated value of the pyloric caeca amount may be displayed at the same time.

Although an ultrasonic inspection unit is represented in FIG. 1, the non-destructive inspection unit 20 may alternatively include an MRI inspection unit or a CT inspection unit instead of the ultrasonic inspection unit. For example, in a case where the non-destructive inspection unit 20 includes an MRI inspection unit, the non-destructive inspection unit 20 may include a superconducting magnet that generates a magnetic field, a transmission unit (high-frequency coil) that transmits an electric wave, a gradient magnetic field coil unit that changes the magnetic field, a reception unit (reception coil) that receives an electric wave from the inside of the fish body, and the like. The MRI inspection unit can obtain a cross-sectional image of the fish by using magnetism, an electromagnetic wave, and the movement of a hydrogen atom. In a case where the non-destructive inspection unit 20 includes a CT inspection unit, the CT inspection unit may include an X-ray tube that irradiates the fish body with X-rays, a detector that detects an X-ray that has passed through the fish body, and a drive unit that rotationally drives the X-ray tube and the detector. As the MRI inspection unit and the CT inspection unit, a commercially available MRI inspection device and a commercially available CT inspection device can be used.

The analysis device 100 estimates the amount of the pyloric caeca based on the third information on the pyloric caeca acquired by the non-destructive inspection. Thus, the pyloric caeca amount of a marine fish can be smoothly estimated without exposing the pyloric caeca. For example, it is possible to estimate a fish's pyloric caeca while its alive. Anesthesia may be applied to the marine fish prior to the non-destructive inspection. By applying anesthesia, an image can be stably acquired by the non-destructive inspection. When the marine fish is returned to seawater immediately after the non-destructive inspection, the marine fish can be awakened from anesthesia. The anesthesia method is not particularly limited as long as the method can fix the fish body for a certain period of time and acquire a stable image by the non-destructive inspection. For example, a marine fish may be immobilized by anesthesia with an anesthetic such as 2-phenoxyethanol or eugenol, or by shock with electric shock, a sound, or a vibration. When the analysis device 100 is used for, for example, farming of a marine fish, it is possible to efficiently provide a marine fish having added value to provide products that meet consumer preferences. Accordingly, the description of the analysis device described above also applies to the description of the farming method below.

### Farming Method

In a method for farming a marine fish according to a first embodiment, an estimated value of the amount of a red muscle contained in a fish body of a marine fish is used as an index in any step of a farming process. The farming process includes any process from hatching to shipping of an adult fish. The estimated value of the amount of the red muscle may be used as an index for sorting a farmed fish (marine fish). That is, the farming method may include a step of sorting the farmed fish based on the estimated value of the amount of the red muscle.

For example, the amount of the red muscle may be estimated at any stage of larva, juvenile, and adult, and the farmed fish (marine fish) may be sorted based on the estimated value. For example, the farmed fish may be sorted into a first group (first marine fish) having a low proportion of the red muscle and a second group (second marine fish) having a higher proportion of the red muscle than that of the first group. Thereafter, when only the first group is continuously farmed, a farmed fish having a lower proportion of the red muscle than that of a conventionally cultured fish can be obtained. In addition, when such sorting is performed a plurality of times at predetermined intervals, it is possible to obtain a farmed fish having a sufficiently lower proportion of the red muscle than that of a conventionally cultured fish. The number of groups to be sorted is not limited to two, and may be three or more. A processed product group of a marine fish obtained through the step of sorting a farmed fish described above (i.e., sorting into fish with lower and higher red muscle contents) can sufficiently reduce the variation in amount of the red muscle. In addition, the amount of the red muscle can be reduced. Such a processed product group of a marine fish has added value.

A fish farming method may include a step of selecting breeding from among the farmed fish based on the estimated value of the amount of the red muscle. When a fish is selected having the lowest proportion of the red muscle from among the farmed fish, the amount of the red muscle of the farmed fish of the next generation can be consistently reduced through the type of feed and other aquaculture means. This makes it possible to efficiently provide a marine fish with added value or the desired red fish muscle content. This breeding of the farming method may be repeated for multiple generations to create a group of farmed fish with a desired range of the amount of the red muscle. Whether or not a group is created by such breeding can be determined by sampling about 10 individuals from the group and measuring the amount of the red muscle.

The amount of the red muscle contained in the fish body of the farmed fish may be estimated by the “Method for Estimating the Amount of Red Muscle” described above. The “Analysis Device for Red Muscle” described above may be used for this estimation. Thus, the descriptions of the “Method for Estimating the Amount of Red Muscle” and the “Analysis Device for Red Muscle” also apply to this farming method.

In a method for farming a marine fish according to a second embodiment, an estimated value of the amount of a pyloric caeca contained in a fish body of a marine fish is used as an index in any step of a farming process. The farming process includes any process from hatching to shipping of an adult fish. The estimated value of the pyloric caeca amount may be used as an index for sorting a farmed fish (marine fish). That is, the farming method may include a step of sorting a farmed fish based on the estimated value of the amount of the pyloric caeca.

For example, the pyloric caeca amount may be estimated at any stage of larva, juvenile, or adult, and the farmed fish (marine fish) may be sorted based on the estimated value. For example, the farmed fish may be sorted into a first group (first marine fish) having a low proportion of the pyloric caeca and a second group (second marine fish) having a higher proportion of the pyloric caeca than that of the first group. Thereafter, when only the first group is continuously farmed, it is possible to obtain a farmed fish having a lower proportion of the pyloric caeca than that of a conventionally cultured fish. In addition, when such sorting is performed a plurality of times at predetermined intervals, it is possible to obtain a farmed fish having a sufficiently lower proportion of the pyloric caeca than that of a conventionally cultured fish. The number of groups to be sorted can be two, three, four, or more. A processed product group of a marine fish obtained through the step of sorting a farmed fish described above can sufficiently reduce the variation in the amount of the pyloric caeca. It is also possible to reduce the amount of the pyloric caeca. Such a processed product group of a marine fish has added value.

The farming method may include a step of selecting breeding from among the farmed fish based on the estimated value of the amount of the pyloric caeca. When breeding fish having the lowest proportion of the pyloric caeca is selected from among the farmed fish, the amount of pyloric caeca of the farmed fish of the next generation can be reduced. This makes it possible to efficiently provide a marine fish with added value. This fish breeding method may be repeated for multiple generations to create a group of farmed fish with the desired range of pyloric caeca. Whether or not a group is created by such breeding can be determined, for example, by sampling about 10 individuals from the group and measuring the amount of the pyloric caeca.

The amount of the pyloric caeca contained in the fish body of the farmed fish may be estimated by “Method for Estimating the Amount of Pyloric caeca” described above. “Analysis Device for Pyloric caeca” described above may be used for this estimation. Thus, the descriptions of “Method for Estimating the Amount of Pyloric caeca” and “Analysis Device for Pyloric caeca” also apply to this farming method.

### Method for Producing Processed Product of Marine Fish

A method for producing a processed product of a marine fish according to a first embodiment includes a step of processing a marine fish in which the amount of the red muscle has been estimated by the above-described method for estimating an amount of red muscle. The marine fish to be processed may be a round fish or a processed product having a lower degree of processing than GG (Gilled and Gutted). In these cases, the red muscle is not exposed, and thus, it is highly useful to estimate the amount of the red muscle. Here, the “processed product with a low degree of processing” refers to a processed product with a small extent of processing. For example, GG has a lower degree of processing than loin. The processing may include, for example, at least one of dissecting, cutting, and cooking. Examples of the processed product of a marine fish obtained by the step include marine raw materials obtained by dissecting or cutting, such as GG, semi-dress, dress, center-cut, fillet, loin, and slice (sashimi). In addition, products obtained by refrigerating, freezing, and/or ripening these products are also included in the processed product of a marine fish. Other examples of the processed product of a marine fish include various processed marine products such as fish meat soaked in a seasoning such as salt, sweet sake, or miso, a paste containing fish meat, canned food, frozen food, and chilled food. The red muscle may not be preferred by some customers because of its color tone. In a market where there are many customers who do not like the red muscle, the red muscle portion may not be processed, and may be removed and discarded depending on the market. Thus, in such a market, a marine fish with a reduced amount of the red muscle is advantageous in that the yield can be increased. On the other hand, there are also customers who want fish meat containing a lot of the red muscle to show fish meat-like appearance or to obtain a variety of colors.

A method for producing a processed product of a marine fish according to a second embodiment includes a step of processing a marine fish farmed by the first embodiment of the above-described farming method. The processing may include, for example, at least one of dissecting, cutting, and cooking. Examples of the processed product of a marine fish obtained by the step are as described above.

A method for producing a processed product of marine fish according to a third embodiment includes a step of processing a marine fish in which the amount of the pyloric caeca has been estimated by the above-described method for estimating the amount of a pyloric caeca. The marine fish to be processed may be a round fish or a processed product having a lower degree of processing than GG (Grilled and Gutted). In these cases, the pyloric caeca is not exposed, and thus, it is highly useful to estimate the amount of the pyloric caeca. Here, the “processed product with a low degree of processing” refers to a processed product with a small extent of processing. For example, GG has a lower degree of processing than loin. The processing may include, for example, at least one of dissecting, cutting, and cooking. Examples of the processed product of a marine fish obtained by the step include marine raw materials obtained by dissecting or cutting, such as GG, semi-dress, dress, center-cut, fillet, loin, and slice (sashimi). In addition, products obtained by refrigerating, freezing, and/or ripening these products are also included in the processed products of marine fish. Other examples of the processed product of a marine fish include various processed marine products such as fish meat soaked in a seasoning, such as salt, sweet sake, or miso, a paste containing fish meat, canned food, frozen food, and chilled food. The pyloric caeca is usually removed and discarded during processing, and thus, a marine fish with a small amount of the pyloric caeca is advantageous in that the yield can be increased.

A method for producing a processed product of a marine fish according to a fourth embodiment includes a step of processing a marine fish farmed by the second embodiment of the farming method described above. The processing may include, for example, at least one of dissecting, cutting, and cooking. Examples of the processed product of a marine fish obtained by the step are as described above.

In the production method according to each of the above embodiments, a marine fish in which the amount of the red muscle or the pyloric caeca has been estimated by the above-described estimation method or farming method is processed, and thus, a processed marine fish product having added value can be efficiently produced flexibly to provide products that meet consumer preferences. For example, a processed marine product having a low proportion of the red muscle and a high proportion of the white muscle can be produced. Alternatively, a processed marine product having a low proportion of the pyloric caeca can be produced.

In the production method according to each of the above embodiments, it is not necessary to sort a marine fish having a small amount of the red muscle or the pyloric caeca after processing. Such an operation is not required, and thus, freshness can be improved. Accordingly, a processed product of a marine fish having a small amount of the red muscle or the pyloric caeca and having high freshness can be obtained. Thus, the freshness of a processed product of a marine fish can be maintained sufficiently high. A K value of a processed product of a marine fish is an index indicating the degree of progress of decomposition of ATP (adenosine triphosphate), and is determined by the following equation. In the following equation, IMP represents inosinic acid, HxR represents inosine, Hx represents hypoxanthine, ADP represents adenosine diphosphate, and AMP represents adenosine monophosphate.

$$K value = \frac{HxR + Hx}{ATP + ADP + AMP + IMP + HxR + Hx} \times 100 \%$$

The smaller the K value, the smaller the proportion of the total amount of HxR and Hx relative to the total amount of ATP and ATP decomposition-related substances. A small (low) K value means that the decomposition of IMP to HxR or Hx has not sufficiently proceeded, that is, a large amount of ATP decomposition-related substances to IMP remains. The K value may be about 35% or less, about 30% or less, about 20% or less, or about 10% or less. The lower limit of the K value may be about 1% or about 5%. The K value may be, for example, about 1 to about 35%, about 1 to about 30%, about 1 to about 20%, about 1 to about 10%, about 5 to about 35%, about 5 to about 30%, about 5 to about 20%, or about 5 to about 10%.

### Identified Product of Marine Fish

An identified product of a marine fish, according to a first embodiment, includes: a fish body or a processed product of a marine fish; and an identification portion including information (second information) indicating that an amount of a red muscle contained in the fish body or the processed product has been estimated by non-destructive inspection. The amount of the red muscle contained in the fish body or the processed product may be estimated by the “Method for Estimating the Amount of Red Muscle” described above. The second information may be related to an estimated value of the amount of the red muscle obtained using the “Analysis Device for Red Muscle” described above. The fish body and the processed product of the marine fish may be a farmed fish obtained by the first embodiment of the farming method described above. Thus, the descriptions of the “Method for Estimating the Amount of Red Muscle”, the “Analysis Device for Red Muscle”, and the “Farming Method” also apply to this identified product of a marine fish. The marine fish may be a round fish or a processed product having a lower degree of processing than that of GG (Grilled and Gutted). In these cases, the red muscle is not exposed, and thus, the second information is highly useful. However, the processed product of a marine fish is not limited to these, and may be any of the examples described above.

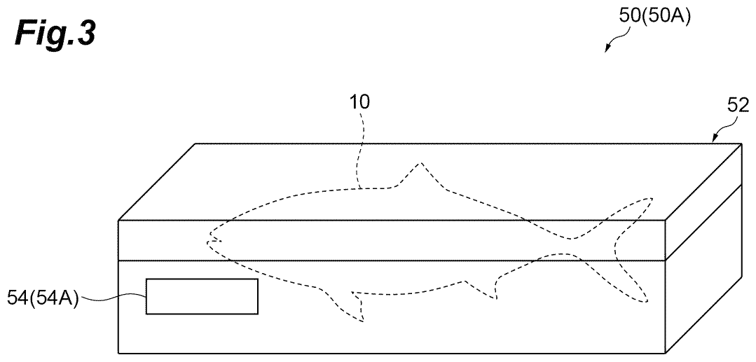

As illustrated in FIG. 3, an example of an identified product 50 of a marine fish includes a container 52, the round fish 10 housed in the container 52, and an identification portion 54. The identification portion 54 includes second information on the estimation result of the amount of the red muscle. The identification portion 54 may be a label or a barcode whose content can be read visually, or may be an IC tag (RF tag) or the like. The identification portion 54 may be a printed letter or a mark directly written on the container 52. Although only one fish body 10 is illustrated in FIG. 3, a plurality of fish bodies or a plurality of processed products may be housed in the container 52. That is, the identified product 50 of a marine fish may include a plurality of fish bodies or a plurality of processed products.

The second piece of information included in the identification portion 54 may be information on an estimated value of the amount of the red muscle contained in the fish body or the processed fish product, which is estimated by non-destructive inspection. Specifically, it may be an estimated value of the weight or volume of the red muscle. However, the second information need not include the estimated value of the amount of the red muscle. For example, the second information may be a symbol indicating that the content is a farmed fish farmed using, as an index, the estimated value of the amount of the red muscle obtained by the non-destructive inspection in the first embodiment of the above-described farming method. In a case where the identified product 50 of a marine fish includes a plurality of fish bodies or a plurality of processed products, the second information may include statistical information such as an average value, a standard deviation, a maximum value, or a minimum value of the amounts of the red muscles.

The second information may include the K value, indicating the freshness of the fish body of the marine fish or the processed product of the marine fish. The equation for determining the K value and the numerical range is as described above. In a case where the identified product of a marine fish includes a plurality of fish bodies or a plurality of processed products, the arithmetic average value of the K values only needs to be within the above-described range.

Although FIG. 3 illustrates the round fish 10, a modified example of the identified product of a marine fish may include one or more processed products of the marine fish in place of the fish body. Examples of the processed products of the marine fish are as described above. The container 52 is not limited to a box-shaped container, and may be a bag body. In this case, the bag body is provided with the identification portion. In another modified example, the fish body or processed product need not be housed in a container. In this case, the identification portion 54 may be attached to the fish body itself or the processed product itself.

Such an identified product of a marine fish has the identification portion 54, and thus, a user or an end user can easily identify a fish body or a processed product having a small amount of red muscle even when the red muscle is not exposed. Accordingly, such an identified product has added value. In addition, in some markets, the red muscle is not preferred and is removed and discarded. In this case, the fish body or processed product having a small amount of the red muscle can increase the yield, and thus has added value.

An identified product of a marine fish according to a second embodiment includes: a fish body or a processed product of a marine fish; and an identification portion including information (fourth information) indicating that the amount of a pyloric caeca contained in the fish body or the processed product has been estimated by non-destructive inspection. The amount of the pyloric caeca contained in the fish body or processed product may be estimated by “Method for Estimating the Amount of Pyloric caeca” described above. The fourth information may be related to an estimated value of the amount of the pyloric caeca obtained using “Analysis Device for Pyloric caeca” described above. The fish body and processed product of a marine fish may be a farmed fish obtained by the above-described farming method. Thus, the descriptions of “Method for Estimating the Amount of Pyloric caeca”, “Analysis Device for Pyloric caeca”, and “Farming Method” also apply to this identified product of a marine fish. The marine fish may be a round fish or a processed product having a lower degree of processing than that of GG (Grilled and Gutted). In these cases, the pyloric caeca is not exposed, and thus, the fourth information is highly useful. However, the processed product of a marine fish is not limited to these, and may be any of the examples described above.

As illustrated in FIG. 3, an example of an identified product 50A of a marine fish includes a container 52, the round fish 10 housed in the container 52, and an identification portion 54A, as illustrated in FIG. 3. The identification portion 54A includes fourth information on the estimation result of the amount of the pyloric caeca. The identification portion 54A may be a label or a barcode whose content can be read visually, or may be an IC tag (RF tag) or the like. The identification portion 54A may be a printed letter or a mark directly written on the container 52. Although only one fish body 10 is illustrated in FIG. 3, a plurality of fish bodies or a plurality of processed products may be housed in the container 52. That is, the identified product 50A of a marine fish may include a plurality of fish bodies or a plurality of processed products.

The fourth information included in the identification portion 54A may be information on an estimated value of the amount of the pyloric caeca contained in the fish body or the processed product estimated by non-destructive inspection. Specifically, it may be an estimated value of the weight or volume of the pyloric caeca. However, the fourth information need not include the estimated value of the pyloric caeca amount. For example, the fourth information may be a symbol indicating that the content is a farmed fish using, as an index, the estimated value of the amount of the pyloric caeca obtained by the non-destructive inspection in the second embodiment of the above-described farming method. In a case where the identified product 50 of a marine fish includes a plurality of fish bodies or a plurality of processed products, the fourth information may include statistical information such as an average value, a standard deviation, a maximum value, or a minimum value of the amounts of the pyloric caeca.

Although FIG. 3 illustrates the round fish 10. A modified example of the identified product of a marine fish may include one or more processed products of a marine fish in place of the fish body. Examples of the processed products of a marine fish are as described above. The container 52 is not limited to a box-shaped container, and may be a bag body. In this case, the bag body is provided with the identification portion. In another modified example, the fish body or processed product need not be housed in a container. In this case, the identification portion 54A may be attached to the fish body itself or the processed product itself.

Such an identified product of a marine fish has the identification portion 54A, and thus, even when the pyloric caeca is not exposed, a user or an end user can easily identify a fish body or a processed product having a small amount of the pyloric caeca. Accordingly, such an identified product has added value. The pyloric caeca is usually removed. In this case, a fish body or processed product with a small amount of the pyloric caeca can have a high yield, and thus has added value.

### Processed Product Group of Marine Fish

A processed product group of a marine fish according to a first embodiment includes a plurality of processed products of a marine fish, and a standard deviation of weight ratios of the red muscles included in the plurality of processed products is 1.5 wt. % or less. Such a processed product group of a marine fish can be obtained by “Method for Producing Processed Product of Marine Fish” described above. Thus, the description of “Method for Producing Processed Product of Marine Fish” described above also applies to this processed product group of a marine fish. The descriptions of the “Method for Estimating the Amount of Red Muscle”, the “Analysis Device for Red Muscle”, the “Farming Method”, and the “Identified Product of Marine Fish” described above also apply to this processed product group of a marine fish. Examples of the processed products of a marine fish are as described above.

The number of processed products included in the processed product group of a marine fish is not particularly limited, and may be 3 or more, 5 or more, or 10 or more. The number of processed products included in the processed product group of a marine fish may be about 1000 or fewer, about 500 or fewer, or about 100 or fewer. The number of processed products included in the processed product group of a marine fish may be, for example, about 3 to about 1000, about 5 to about 1000, about 10 to about 1000, about 3 to about 500, about 3 to about 100, or about 5 to about 500. The processed product group of a marine fish may be housed in the container 52 or a bag body, as illustrated in FIG. 3, or need not be housed.

In the processed product group of a marine fish of the present embodiment, the variation in weight of the red muscle is sufficiently reduced. Thus, the processed product group has high quality and reliability and has added value. From the viewpoint of further reducing the variation in weight of the red muscle, the standard deviation of the weight ratios of the red muscles contained in the plurality of processed products may be about 1.50 wt. % or less, about 1.00 wt. % or less, about 0.50 wt. % or less, about 0.40 mass % or less, about 0.30 mass % or less, about 0.20 wt. % or less, or about 0.10 wt. % or less. A standard deviation of weight ratios of the red muscles contained in the plurality of processed products may be about 0.001 wt. % or more. The standard deviation of the weight ratios may be, for example, about 0.001 wt. % to about 1.50 wt. %, about 0.001 wt. % to about 1.00 wt. %, about 0.001 wt. % to about 0.50 wt. %, about 0.001 wt. % to about 0.40 wt. %, about 0.001 wt. % to about 0.30 wt. %, about 0.001 wt. % to about 0.20 wt. %, or about 0.001 wt. % to about 0.10 wt. %.

The processed product group of a marine fish may be obtained by processing a fish body of a round fish or a processed product having a lower degree of processing than GG. The processed product group of a marine fish may be obtained by processing the first marine fish sorted in the step of sorting, the farming method described above. Such a processed product group of a marine fish can reduce the amount of the red muscle. This makes it possible to maintain the freshness sufficiently high. The arithmetic average value of the K values of the processed product group of a marine fish may be as described above.

The processed product group of a marine fish may include an identification portion including second information on an estimated value of the amount of the red muscle. The second information and the identification portion may be as described above. This makes it possible to predict the amount of a red muscle based on the second information even when the red muscle is not exposed. Thus, the processed product group has high quality and reliability and has added value.

A processed product group of a marine fish according to a second embodiment includes a plurality of processed products of a marine fish, and a standard deviation of weight ratios of pyloric caeca contained in the plurality of processed products is 10.0 wt. % or less. Such a processed product group of a marine fish can be obtained by “Method for Producing Processed Product of Marine Fish” described above. Thus, the description of “Method for Producing Processed Product of Marine Fish” also applies to this processed product group of a marine fish. The descriptions of “Method for Estimating the Amount of Pyloric caeca”, “Analysis Device for Pyloric caeca”, “Farming Method”, and “Identified Product of Marine Fish” described above also apply to this processed product group of a marine fish. Examples of the processed product of a marine fish are as described above.

The number of processed products included in the processed product group of a marine fish is not particularly limited, and may be 3 or more, 5 or more, or 10 or more. The number of processed products included in the processed product group of a marine fish may be 1000 or fewer, 500 or fewer, or 100 or fewer (or any digit between 3 and 1000). The number of processed products included in the processed product group of a marine fish may be, for example, about 3 to about 1000, about 5 to about 1000, about 10 to about 1000, about 3 to about 500, about 3 to about 100, or about 5 to about 500. The processed product group of a marine fish may be housed in the container 52 or a bag body, as illustrated in FIG. 3, or need not be housed.

In the processed product group of a marine fish of the present embodiment, the variation in weight of the pyloric caeca is sufficiently reduced. Thus, the processed product group has high quality and reliability and has added value. From the viewpoint of further reducing the variation in weight of the pyloric caeca, the standard deviation of the weight ratios of the pyloric caeca contained in the plurality of processed products may be about 10.0 wt. % or less, about 7.0 wt. % or less, about 5.0 wt. % or less, about 3.0 wt. % or less, about 1.5 wt. % or less, about 1.0 wt. % or less, about 0.70 wt. % or less, about 0.50 mass % or less, about 0.30 mass % or less, about 0.20 wt. % or less, or about 0.10 wt. % or less. The standard deviation of the weight ratios of the pyloric caeca contained in the plurality of processed products may be about 0.001 wt. % or more. The standard deviation of the weight ratios may be, for example, about 0.001 wt. % to about 10.0 wt. %, about 0.001 wt. % to about 7.0 wt. %, about 0.001 wt. % to about 5.0 wt. %, about 0.001 wt. % to about 3.0 wt. %, about 0.001 wt. % to about 2.0 wt. %, about 0.001 wt. % to about 1.0 wt. %, about 0.001 wt. % to about 0.70 wt. %, about 0.001 wt. % to about 0.50 wt. %, about 0.001 wt. % to about 0.30 wt. %, about 0.001 wt. % to about 0.20 wt. %, or about 0.001 wt. % to about 0.10 wt. %.

The processed product group of a marine fish may be obtained by processing a fish body of a marine round fish or a processed product having a lower degree of processing than GG. The processed product group of a marine fish may be obtained by processing the first marine fish sorted in the step of sorting using the farming method described above. Such a processed product group of a marine fish can reduce the amount of the pyloric caeca. The method can make possible a means to reduce the pyloric caeca amount, thereby increasing the yield. The arithmetic average value of the K values of the processed product group of a marine fish may be as described above.

The processed product group of a marine fish may include an identification portion including fourth information on the estimated of pyloric caeca. The fourth information and the identification portion may be as described above. This makes it possible to predict the amount of a pyloric caeca based on a fourth piece of information, even when the pyloric caeca is not exposed. Thus, the processed product group has high quality reliability and has added value.

Although the embodiments of the present disclosure have been described above, the present disclosure is not limited to the above embodiments. The present disclosure includes the following [1] to [38].

[1] A method for estimating the red fish muscle amount, the method comprising: an information acquisition step of acquiring information on a red muscle in a fish body of a marine fish by non-destructive inspection; and an estimation step of estimating an amount of the red muscle contained in the fish body based on the information.

[2] The estimation method according to [1], wherein the information acquired in the information acquisition step includes a cross-sectional area of the red muscle determined from a cross-sectional image of the fish body, and the amount of the red muscle is estimated based on the cross-sectional area in the estimation step.

[3] The estimation method according to [1] or [2], further comprising a weight measurement step of measuring the weight of the fish body, wherein in the estimation step, the weight of the red muscle is estimated based on the weight of the fish body and the cross-sectional area of the red muscle.

[4] The estimation method according to any one of [1] to [3], wherein the non-destructive inspection uses at least one selected from the group consisting of ultrasonic inspection, MRI inspection, and CT inspection.

[5] The estimation method according to any one of [1] to [4], wherein the information is acquired from a transverse section image between the fourth vertebra to the twenty-second vertebra or a transverse section image between a second dorsal fin beginning portion and a vicinity of an anus, of the marine fish.

[6] The estimation method according to any one of [1] to [5], wherein the information is acquired from a transverse section image between an eleventh vertebra to a seventeenth vertebra or a transverse section of a vicinity of the anus.

[7] A method for producing a processed product of a marine fish, the method comprising a step of processing the marine fish in which the amount of the red muscle has been estimated by the estimation method according to any one of [1] to [6].

[8] A method for farming a marine fish, the method comprising using an estimated value of an amount of a red muscle contained in a fish body of a marine fish as an index in any step of a farming process.

[9] The method for farming a marine fish according to [8], the method further comprising a step of sorting the marine fish based on the estimated value.

[10] The method for farming a marine fish according to [8] or [9], wherein the estimated value is a value estimated based on an image of the red muscle in the fish body acquired by non-destructive inspection.

[11] A method for producing a processed product of a marine fish, the method comprising a step of processing the marine fish farmed by the farming method according to any one of [8] to [10] above.

[12] An analysis device comprising: a non-destructive inspection unit that acquires information on a red muscle in a marine fish body of a marine fish by non-destructive inspection; and a red muscle estimation unit that estimates an amount of the red muscle contained in the marine fish body of the marine fish based on the information.

[13] The analysis device according to [12], wherein the non-destructive inspection unit includes at least one selected from the group consisting of an ultrasonic inspection unit, an MRI inspection unit, and a CT inspection unit.

[14] An identified product of a marine fish, comprising: a fish body or a processed product of a marine fish; and an identification portion including information indicating that an amount of a red muscle contained in the fish body or the processed product has been estimated by non-destructive inspection.

[15] A processed product group of a marine fish, comprising a plurality of processed products of a marine fish, wherein a standard deviation of weight ratios of red muscles contained in the plurality of processed products is 1.5 wt. % or less.

[16] The processed product group of a marine fish, according to [15], has a K value of 35% or less.

[17] The processed product group of a marine fish according to [15] or [16], further comprising an identification portion including information on an estimated value of a weight of a red muscle contained in each of the plurality of processed products.

[18] The processed product group of a marine fish according to any one of [15] to [17], being obtained by processing a fish body of the marine round fish or a processed product of the marine round fish having a lower degree of processing than GG.

[19] A processed product group of a marine fish obtained by processing a first marine fish that is a part of the marine fish sorted in the step of sorting the marine fish in the farming method according to [9] or [10] above.

[20] A method for estimating an amount of a pyloric caeca, the method comprising: an information acquisition step of acquiring information on a pyloric caeca in a fish body of a marine fish by non-destructive inspection; and an estimation step of estimating an amount of the pyloric caeca contained in the fish body based on the information.

[21] The estimation method according to [20], wherein the information acquired in the information acquisition step includes a cross-sectional area of the pyloric caeca determined from a cross-sectional image of the fish body, and in the estimation step, the amount of the pyloric caeca is estimated based on the cross-sectional area.

[22] The estimation method according to [21], further comprising a weight measurement step of measuring the weight of the fish body, wherein in the estimation step, a weight of the pyloric caeca is estimated based on the weight of the fish body and the cross-sectional area of the pyloric caeca.

[23] The estimation method according to any one of [20] to [22], wherein the non-destructive inspection uses at least one selected from the group consisting of ultrasonic inspection, MRI inspection, and CT inspection.

[24] The estimation method according to any one of [20] to [23], wherein the information is acquired from a transverse section image between a second vertebra to an eleventh vertebra of the marine fish or a transverse section image between a pectoral fin beginning portion and a second dorsal fin beginning portion.

[25] The estimation method according to any one of [20] to [24], wherein the information is acquired from a transverse section image between the fourth vertebra to the ninth vertebra of the marine fish or a transverse section image of a vicinity of a pelvic fin root.

[26] A method for producing a processed product of a marine fish, the method comprising a step of processing the marine fish in which the amount of the pyloric caeca has been estimated by the estimation method according to any one of [20] to [25] above.

[27] A method for farming a marine fish, the method comprising using an estimated value of an amount of a pyloric caeca contained in a fish body of a marine fish as an index in any step of a farming process.

[28] The method for farming a marine fish according to [27], the method further comprising a step of sorting the marine fish based on the estimated value.

[29] The method for farming a marine fish according to [27] or [28], wherein the estimated value is a value estimated based on an image of the pyloric caeca in the fish body acquired by non-destructive inspection.

[30] A method for producing a processed product of a marine fish, the method comprising a step of processing the marine fish farmed by the farming method according to any one of [27] to [29].

[31] An analysis device comprising:
a non-destructive inspection unit that acquires information on a pyloric caeca in a fish body of a marine fish by non-destructive inspection; and
a pyloric caeca estimation unit that estimates an amount of the pyloric caeca contained in the fish body based on the information.

[32] The analysis device according to [31], wherein the non-destructive inspection unit includes at least one selected from the group consisting of an ultrasonic inspection unit, an MRI inspection unit, and a CT inspection unit.

[33] An identified product of a marine fish, comprising: a fish body or a processed product of a marine fish; and an identification portion including information indicating that an amount of a pyloric caeca contained in the fish body or the processed product has been estimated by non-destructive inspection.

[34] A processed product group of a marine fish, comprising a plurality of processed products of a marine fish, wherein a standard deviation of weight ratios of pyloric caeca contained in the plurality of processed products is 10.0 wt. % or less.

[35] The processed product group of a marine fish according to [34], having a K value of 35% or less.

[36] The processed product group of a marine fish according to [34] or [35], further comprising an identification portion including information on an estimated value of a weight of a pyloric caeca included in each of the plurality of processed products.

[37] The processed product group of a marine fish according to any one of [34] to [36], being obtained by processing a fish body of a marine round fish or a processed product of marine fish having a lower degree of processing than GG.

[38] A processed product group of a marine fish, being obtained by processing a first marine fish that is a part of the marine fish, the first marine fish being sorted in the step of sorting the marine fish in the farming method according to [28] above.

[39] A method for estimating an amount of a pyloric caeca, the method comprising the steps of: acquiring information on a pyloric caeca in a fish body of a marine fish by operating a non-destructive inspection unit; and using a calculation unit, estimating an amount of the pyloric caeca contained in the fish body based on the acquired information.

[40] A method for farming a marine fish, the method comprising the steps of: determining an estimated value of an amount of a pyloric caeca contained in a marine fish body, and adjusting a farming condition for the marine fish based on the estimated value.

[41] An identified product of a marine fish, comprising: a fish body or a processed product of a marine fish; and an identification portion including information indicating that an amount of a pyloric caeca contained in the fish body or the processed product, wherein the amount of the pyloric caeca has been estimated by non-destructive inspection, and wherein the identification portion is a machine-readable medium configured to cause a computer system to perform a specific action related to the fish body or the processed product when read by the computer system.

### EXAMPLES

Although the content of the present disclosure is explained below in further detail with reference to Examples, the present disclosure is not limited to Examples described below.

### Example 1: Relationship (1) Between Cross-Sectional Area (Measured Value) of Red Muscle and Weight of Red Muscle

The following examination was performed to identify a position where a cross-sectional area of a red muscle and a weight of the red muscle correlate with each other. Eight yellowtails randomly sampled on the sea were sliced at three sites, and cross-sectional areas of a red muscle and a white muscle in the three transverse sections were measured. The cut sites were a pelvic fin beginning portion (a), a second dorsal fin beginning portion (b), and an anus (c). The white muscle and the red muscle were cut out from the cut yellowtail and weighed. A body weight, fork length, condition factor, sex, weight of the white muscle, and a weight of the red muscle of the yellowtail used for the measurement were as shown in Table 1. The condition factor was calculated by the following calculation equation. The cross-sectional areas of the red muscle and the white muscle in each of the transverse sections of (a), (b), and (c) were as shown in Table 2.

$$Condition factor = \frac{weight ( g )}{[ {\{ fork length ( cm ) \}}^{3} ]} \times 1000$$

TABLE 1

| | Body | Fork | | | Weight of | Weight of |
|---|---|---|---|---|---|---|
| | Weight | length | Condition | | white muscle | red muscle |
| n | [g] | [cm] | factor | Sex | [g] | [g] |
| 1 | 3251 | 63.2 | 12.9 | Male | 597.0 | 121.5 |
| 2 | 3420 | 64.2 | 12.9 | Male | 766.5 | 136.5 |
| 3 | 3125 | 62.4 | 12.9 | Male | 668.5 | 111.5 |
| 4 | 3185 | 63.0 | 12.7 | Male | 673.0 | 136.0 |
| 5 | 4235 | 71.5 | 11.6 | Female | 810.0 | 181.0 |
| 6 | 3010 | 60.9 | 13.3 | Male | 616.5 | 120.5 |
| 7 | 3060 | 62.0 | 12.8 | Female | 612.0 | 115.0 |
| 8 | 3330 | 64.8 | 12.2 | Female | 631.5 | 139.0 |

TABLE 2

| | |
|---|---|
| Cross-sectional | Cross-sectional |
| area of white muscle | area of red muscle |

| | (a) | (b) | (c) | (a) | (b) | (c) |
|---|---|---|---|---|---|---|
| n | [cm²] | [cm²] | [cm²] | [cm²] | [cm²] | [cm²] |
| 1 | 30.987 | 34.311 | 23.931 | 3.494 | 5.134 | 5.545 |
| 2 | 30.755 | 32.880 | 27.458 | 3.742 | 4.960 | 5.706 |
| 3 | 30.269 | 29.870 | 22.697 | 2.263 | 4.534 | 4.364 |
| 4 | 27.173 | 31.656 | 23.057 | 3.703 | 4.723 | 5.631 |
| 5 | 33.345 | 35.328 | 28.681 | 3.227 | 5.433 | 6.228 |
| 6 | 32.098 | 29.421 | 25.196 | 4.654 | 3.079 | 5.452 |
| 7 | 25.411 | 31.043 | 23.744 | 2.560 | 4.437 | 4.527 |
| 8 | 30.914 | 34.020 | 21.509 | 2.938 | 4.330 | 4.921 |

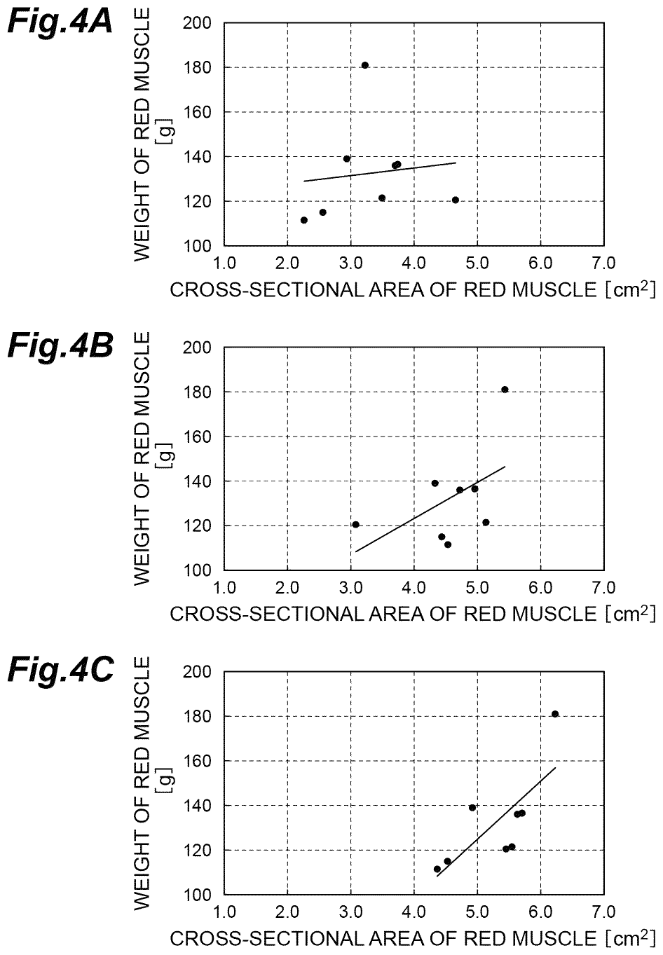

FIG. 4A is a graph showing a relationship between a cross-sectional area of the red muscle and a weight of the red muscle in the pelvic fin beginning portion (position A in FIG. 2). FIG. 4B is a graph showing a relationship between a cross-sectional area of the red muscle and a weight of the red muscle in the second dorsal fin beginning portion (position B in FIG. 2). FIG. 4C is a graph showing a relationship between the cross-sectional area of the red muscle and the weight of the red muscle in the anus (position of the probe 22 in FIG. 2). When the results of FIG. 4A, FIG. 4B, and FIG. 4C are compared, FIG. 4C shows the strongest correlation. Using the data shown in Table 2, a Pearson's product-moment correlation coefficient (PCC, hereinafter referred to as “product-moment correlation coefficient”) of each of the cut sites (a), (b), and (c) was determined. The results are shown in Table 3. As a result, the product-moment correlation coefficient between the cross-sectional area of the red muscle and the weight of the red muscle measured in the transverse section passing through the second dorsal fin beginning portion (b) and the anus (c) exceeded 0.4, and it was confirmed that there was a certain degree of correlation. The product-moment correlation coefficient between the cross-sectional area of the red muscle and the weight of the red muscle measured in the transverse section passing through the anus (c) exceeded 0.7, and it was confirmed that there was a strong correlation.

TABLE 3

| | (a) | (b) | |
|---|---|---|---|
| | Pelvic fin | Second dorsal | |
| | beginning | fin beginning | (c) |
| Cut site | portion | portion | Anus |
| Product- | 0.12 | 0.52 | 0.75 |
| moment | | | |
| correlation | | | |
| coefficient | | | |

### Example 2: Relationship (2) Between Cross-Sectional Area (Measured Value) of Red Muscle and Weight of Red Muscle

Ten farm raised yellowtails randomly sampled on the sea were sliced at eight sites, and cross-sectional areas of the red muscle and the white muscle in each of the eight transverse sections were measured. The cut sites were as follows. The names of the respective parts were as described in ATLAS OF FISH ANATOMY (Akira Ochiai, Midori Shobo, 1987, p. 192).
(i) Between the second vertebra and the fourth vertebra
(ii) Between the fourth vertebra and the seventh vertebra
(iii) Between the seventh vertebra and the ninth vertebra
(iv) Between the ninth vertebra and the eleventh vertebra
(v) Between the eleventh vertebra and the fourteenth vertebra
(vi) Between the fourteenth vertebra and the seventeenth vertebra
(vii) Between the seventeenth vertebra and the nineteenth vertebra
(viii) Between the nineteenth vertebra and the twenty-second vertebra

The white muscle and the red muscle were cut out from each of the cut yellowtails and weighed. The body weight, fork length, condition factor, weight of the white muscle, and weight of the red muscle of each of the yellowtails used for the measurement were as shown in Table 4. The cross-sectional area of the red muscle in each cross-section is shown in Table 5. Using the data shown in Table 5, the product-moment correlation coefficient between the cross-sectional area and the weight of each red muscle was determined. The results are shown in Table 6.

As shown in Table 6, the product-moment correlation coefficient exceeded 0.4 in the range of (ii) to (viii), which was from the fourth vertebra to the twenty-second vertebra, and it was confirmed that there was a certain degree of correlation. In the range of (v) to (vi), which was from the eleventh vertebra to the seventeenth vertebra, the product-moment correlation coefficient exceeded 0.7, and it was confirmed that there was a strong correlation. Note that the anus is between (v) and (vi). The white muscle is present from the back side to the ventral side, unlike the red muscle located in the central portion of the lateral side of the fish's body. Thus, the influence of the fish body size is highly likely to be greatly reflected, and it is difficult to accurately measure the entire area of the white muscle. In contrast, the area of the red muscle located in the central portion of the lateral side of the fish's body can be measured with higher accuracy than the area of the white muscle. Thus, in each section from the fourth vertebra to the twenty-second vertebra, a higher product-moment correlation coefficient was obtained than in the other sections. From the results of Table 3 and Table 6, it was considered that a strong correlation was similarly achieved even when the area was measured by acquiring the transverse section using another method, for example, a non-destructive inspection method, such as capturing an echo image.

TABLE 4

| | | | | Weight | Weight |
|---|---|---|---|---|---|
| | Body | Fork | | of white | of red |
| | Weight | length | Condition | muscle | muscle |
| n | [g] | [cm] | factor | [g] | [g] |
| 1 | 537 | 32.5 | 15.6 | 103.0 | 18.1 |
| 2 | 607 | 34.0 | 15.4 | 123.1 | 18.9 |
| 3 | 423 | 30.4 | 15.1 | 84.0 | 14.5 |
| 4 | 475 | 31.8 | 14.8 | 96.8 | 16.9 |
| 5 | 399 | 30.6 | 13.9 | 80.4 | 13.3 |
| 6 | 481 | 31.7 | 15.1 | 92.1 | 15.9 |
| 7 | 534 | 33.0 | 14.9 | 99.4 | 19.8 |
| 8 | 568 | 32.9 | 15.9 | 114.6 | 18.4 |
| 9 | 518 | 32.6 | 15.0 | 105.7 | 17.3 |
| 10 | 625 | 33.8 | 16.2 | 128.8 | 19.9 |

TABLE 5

| | Cross-sectional area (red muscle) | | | | | | | |
|---|---|---|---|---|---|---|---|---|
| | (i) | (ii) | (iii) | (iv) | (v) | (vi) | (vii) | (viii) |
| n | [cm²] | [cm²] | [cm²] | [cm²] | [cm²] | [cm²] | [cm^{2]} | [cm^{2]} |
| 1 | 1.104 | 1.068 | 1.223 | 1.103 | 1.064 | 0.882 | 0.644 | 0.403 |
| 2 | 0.911 | 1.208 | 1.093 | 1.189 | 1.133 | 1.021 | 0.979 | 0.588 |
| 3 | 0.464 | 0.893 | 0.858 | 0.815 | 0.884 | 0.829 | 0.594 | 0.371 |
| 4 | 0.976 | 1.107 | 1.141 | 0.953 | 1.033 | — | 0.668 | 0.421 |
| 5 | 0.739 | 0.800 | 0.802 | 0.910 | 0.726 | 0.537 | 0.465 | 0.216 |
| 6 | 0.761 | 0.775 | 0.923 | 0.950 | 0.937 | 0.808 | 0.459 | 0.286 |
| 7 | 0.732 | 0.765 | 0.911 | 0.912 | 0.990 | 0.862 | 0.575 | 0.341 |
| 8 | 0.733 | 1.136 | 0.968 | 1.038 | 1.022 | 0.887 | 0.583 | 0.339 |
| 9 | 0.484 | 0.783 | 0.868 | 1.025 | 0.909 | 0.938 | 0.676 | 0.431 |
| 10 | 0.849 | 1.117 | 1.126 | 1.26 | 1.202 | 0.981 | 0.696 | 0.316 |

In Table 5, “—” indicates that the measurement was not performed.

TABLE 6

| Cut site | (i) | (ii) | (iii) | (iv) | (v) | (vi) | (vii) | (viii) |
|---|---|---|---|---|---|---|---|---|
| Product-moment | 0.37 | 0.46 | 0.58 | 0.68 | 0.86 | 0.80 | 0.53 | 0.42 |
| correlation coefficient | | | | | | | | |

### Example 3: Capturing of Transverse Sectional Echo Image by Ultrasonic Inspection Device

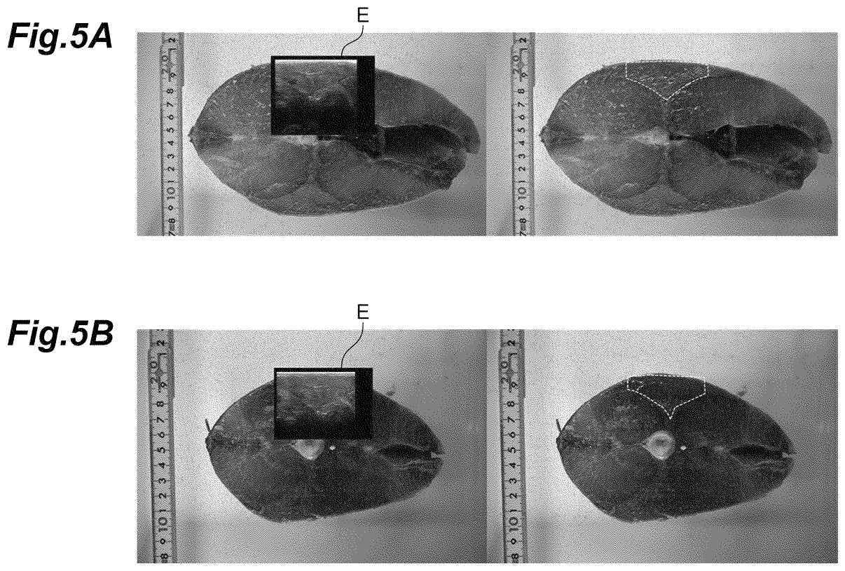

Anesthesia was applied to a farm raised yellowtail randomly sampled on the sea, and an echo image of a transverse section of the fish body was taken using a commercially available ultrasonic inspection device shown below. After the photographing, the yellowtail was cut at the site where the transverse section echo image was taken, and the photograph of the transverse section echo image was compared with the actual photograph of the actual cut surface. FIG. 5A and FIG. 5B show the results. FIG. 5A is a photograph of the vicinity of the dorsal fin root, and FIG. 5B is a photograph of the vicinity of the anus. In both FIG. 5A and FIG. 5B, the left photograph shows a transverse section echo image E of the red muscle overlapped with the photograph of the cross-sectional image, and the right photograph shows the red muscle observed in the photograph of the actual cut surface surrounded by a dotted line. From these results, it was confirmed that the epithelial side can be determined as the red muscle with a boundary line appearing white in the muscle as a boundary in the transverse section echo image by the ultrasonic inspection device.

### Ultrasonic Inspection Device

Model name: MyLabOne VET
Manufacturer: Esaote Europe B.V. (The Netherlands)
Medical device approval number: 24 Animal drug 2892
Probe: Linear-type SV3513
Focal length: 0 to 150 mm
B-mode frequency: 6.0, 8.0, 10.0 MHz

### Example 4: Relationship (1) Between the Cross-Sectional Area of Red Muscle by Transverse Section Echo Image and the Weight of Red Muscle

Anesthesia was applied to ten yellowtails randomly sampled from the sea, and the ten yellowtails were placed sideways on a measurement table. The body weight and sex of each of the yellowtails used for the measurement are shown in Table 7. In the same manner as in Example 3, an echo image of a transverse section passing through the anus of each fish's body was taken, and a portion of the red muscle was specified. The cross-sectional area of the red muscle was determined by performing image processing on the photograph of the captured transverse section echo image using image processing software (Image J). The cross-sectional areas determined from the transverse section echo images are shown in Table 7. After ultrasonic inspection, each fish body was disassembled to take out the red muscle and the white muscle, and the weights of the red muscle and the white muscle were measured using a scale. The measurement results are shown in Table 7.

TABLE 7

| | | | Cross- | | |
|---|---|---|---|---|---|
| | | | sectional | Weight | Weight |
| | Body | | area of red | of white | of red |
| | Weight | | muscle | muscle | muscle |
| n | [g] | Sex | [cm²] | [g] | [g] |
| 1 | 690 | Female | 1.313 | 138.83 | 26.30 |
| 2 | 590 | Male | 1.151 | 119.25 | 23.16 |
| 3 | 576 | Female | 1.076 | 112.48 | 22.80 |
| 4 | 593 | Male | 1.102 | 115.43 | 21.06 |
| 5 | 684 | Male | 1.267 | 134.40 | 27.95 |
| 6 | 659 | Female | 1.230 | 125.99 | 24.15 |
| 7 | 702 | Male | 1.387 | 141.42 | 28.93 |
| 8 | 556 | Male | 0.912 | 110.45 | 16.90 |
| 9 | 551 | Male | 0.958 | 100.67 | 19.26 |
| 10 | 545 | Male | 0.977 | 103.89 | 18.38 |
| Average | 615 | — | 1.137 | 120.28 | 22.89 |
| value | | | | | |

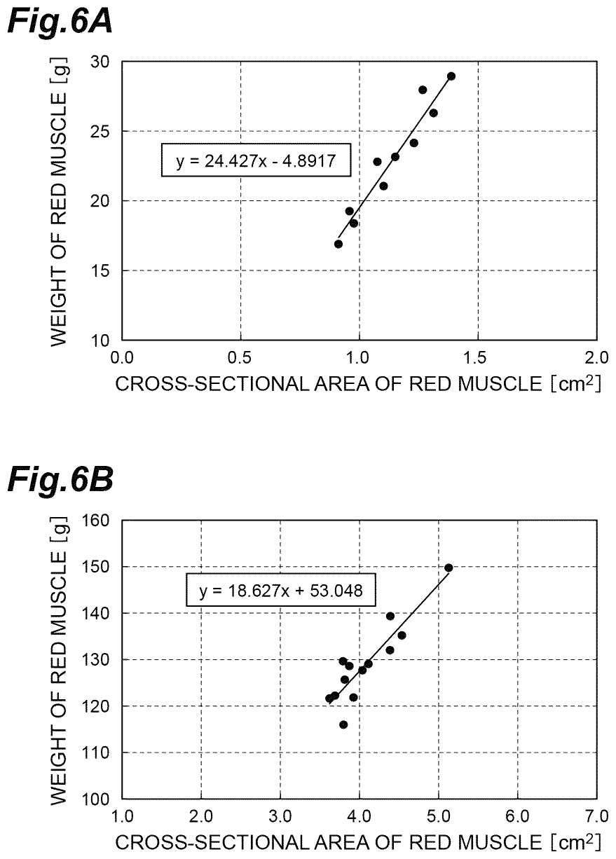

FIG. 6A is a graph showing a relationship between the cross-sectional area of the red muscle determined from the transverse section echo image and the weight of the red muscle actually measured, shown in Table 7. As shown in FIG. 6A, the cross-sectional area of the red muscle determined from the transverse section echo image and the weight of the red muscle actually measured showed a statistically significant correlation. Using the data shown in Table 7, the product-moment correlation coefficient between the cross-sectional area of the red muscle determined from the transverse section echo image and the weight of the red muscle actually measured was determined. As a result, the product-moment correlation coefficient was 0.97. This value was higher than the product-moment correlation coefficient shown in Tables 3 and 6. In addition, the product-moment correlation coefficient between the cross-sectional area of the red muscle and the weight of the white muscle was determined and is shown in Table 7. As a result, the product-moment correlation coefficient was 0.96. From this result, it has been found that the weight of the white muscle can also be estimated with high accuracy from the cross-sectional area of the red muscle. It was considered that atrophy or deformation of tissues of the red muscle and the white muscle due to the cutting for the purpose of fish meat processing was small in the non-destructive inspection, and thus, an estimation with higher accuracy was possible.

### Example 5: Relationship (2) Between the Cross-Sectional Area of Fish Red Muscle by Transverse Section Echo Image and the Weight of Red Muscle

Using 13 yellowtails randomly sampled from the sea (i.e., wild yellowtail), the correlation between the cross-sectional area of the red muscle determined from the transverse section echo image and the weight of the red muscle actually measured was examined in the same manner as in Example 4. The body weight, fork length, condition factor, and sex of each of the yellowtails used for the measurement are shown in Table 8. The cross-sectional area determined from the transverse section echo image and the weights of the red muscle and the white muscle that were actually measured are shown in Table 8.

TABLE 8

| | | | | | Cross-sectional | | |
|---|---|---|---|---|---|---|---|
| | Body | Fork | | | area of red | Weight of | Weight of |
| | Weight | length | Condition | | muscle | white muscle | red muscle |
| n | [g] | [cm] | factor | Sex | [cm²] | [g] | [g] |
| 1 | 3230 | 52.4 | 22.4 | Male | 3.871 | 610.99 | 128.63 |
| 2 | 2890 | 50.8 | 22.0 | Male | 3.814 | 545.18 | 125.68 |
| 3 | 3015 | 50.6 | 23.3 | Male | 4.113 | 627.96 | 129.06 |
| 4 | 2720 | 51.1 | 20.4 | Female | 3.690 | 519.97 | 122.27 |
| 5 | 2810 | 50.7 | 21.6 | Male | 3.791 | 543.65 | 129.64 |
| 6 | 3095 | 52.0 | 22.0 | Female | 4.037 | 642.44 | 127.69 |
| 7 | 3005 | 52.4 | 20.9 | Male | 4.391 | 566.48 | 139.33 |
| 8 | 2530 | 48.2 | 22.6 | Female | 3.622 | 479.95 | 121.66 |
| 9 | 2830 | 50.5 | 22.0 | Male | 4.537 | 546.99 | 135.20 |
| 10 | 2885 | 51.0 | 21.7 | Female | 4.386 | 555.87 | 132.02 |
| 11 | 3285 | 51.8 | 23.6 | Male | 5.127 | 608.10 | 149.73 |
| 12 | 2645 | 49.7 | 21.5 | Female | 3.798 | 485.66 | 116.00 |
| 13 | 2470 | 49.5 | 20.4 | Male | 3.924 | 451.35 | 121.85 |
| Average | 2878 | 50.8 | 21.9 | — | 4.085 | 552.66 | 129.14 |
| value | | | | | | | |

FIG. 6B is a graph showing a relationship between the cross-sectional area of the red muscle determined from the transverse section echo image and the weight of the red muscle actually measured, shown in Table 8. As shown in FIG. 6B, the cross-sectional area of the red muscle determined from the transverse section echo image and the weight of the red muscle actually measured showed a statistically significant correlation. Using the data shown in Table 8, the product-moment correlation coefficient and p-value for the Pearson's product-moment correlation were determined for the relationship between the cross-sectional area of the red muscle obtained from the transverse section echo image and the actually measured weight of the red muscle. As a result, the product-moment correlation coefficient was 0.90, and the p-value was <0.0001. This correlation coefficient value was also higher than the product-moment correlation coefficients shown in Tables 3 and 6.

As shown in the column of body weight in each of Tables 7 and 8, the body weights of the fish bodies used in Examples 4 and 5 are significantly different. From the analysis of this data, it has been confirmed that the weight of the red muscle can be estimated with high accuracy based on the cross-sectional area of the red muscle determined from the transverse section echo image, regardless of the body weight of the fish body. FIG. 6A and FIG. 6B show correlation equations calculated based on the respective data. When the correlation equation is set for each fish's body weight, the weight of the red muscle can be estimated with higher accuracy.

### Example 6: Standard Deviation of Weight Ratios of Red Muscles

The cross-sectional area of the red muscle of each of 33 yellowtails (5 groups) randomly sampled from the sea was measured using the body weight and the echo image in the same manner as in Example 4. In addition, as in Example 4, after the ultrasonic inspection, each fish body was disassembled to take out the red muscle and the white muscle, and the weights of the red muscle and the white muscle were measured using a scale. The results were as shown in Tables 9 and 10. The weights of the yellowtails were from 399 g to 3285 g, the average value of the weights was 1476 g, and the standard deviation was 1141 g. The cross-sectional areas of the red muscles of the yellowtails sampled were 0.726 cm² to 5.127 cm², the average value of the weights of the red muscles was 63 g, and the standard deviation was 54 g. The amount of red muscle tissue estimation score of each fish was determined to be 2.17 for the maximum value, 1.20 for the minimum value, 1.70 for the average value, 1.79 for the median, and 0.25 for the standard deviation. Note that the amount of red muscle tissue estimation score was calculated by the following equation. Tables 9 and 10 show the 33 yellowtails classified into five groups based on the value of the amount of red muscle tissue estimation score. The “amount of red muscle tissue estimation score” is calculated as shown below.

$$Amount of red muscle tissue estimation score = \frac{cross - sectional area of red muscle ( {cm}^{3} )}{body weight ( g )} \times 1000$$

Group 4 in Table 10 shows individuals (yellowtails) with an amount of red muscle tissue estimation score within the median ±5%. Note that % of the amount of red muscle tissue estimation score is a value obtained by dividing the difference between the maximum value and the minimum value of the amount of red muscle tissue estimation score by 100. The number of individuals belonging to Group 4 was 14.

For all 33 individuals, the weight of the red muscle was measured in the same manner as in Example 1, and the weight ratio (%) of the red muscle to the body weight was determined. The weight ratio of the red muscle to the body weight was 3.04 wt. % to 4.93 wt. %, the average value of the weight ratio of the red muscle to the body weight was 3.91 wt. %, and the standard deviation was 0.56 wt. %. On the other hand, the weight ratios of the red muscle to the body weight of the 14 individuals of Group 4 collected based on the amount of red muscle tissue estimation score ranged between 3.11 wt. % to 4.12 wt. %. The average value of the weight ratios of the red muscles to the body weights was 3.60 wt. %, and the standard deviation was 0.33 wt. %. For the other groups of fish measured, the average value and standard deviation of the weight ratios of the red muscles to the body weights also were determined. As a result, the standard deviation of each group was smaller than the value (0.56 wt. %) for all 33 individuals. That is, by using the amount of red muscle tissue estimation score, it was possible to obtain a group with a reduced variation in the weight ratio of the red muscle to the body weight in a non-destructive state.

TABLE 9

| | | | | | Cross- | Weight ratio of | Amount of red |
|---|---|---|---|---|---|---|---|
| | | Body | Weight of | Weight of | sectional area | red muscle to | muscle tissue |
| Group | | Weight | white muscle | red muscle | of red muscle | body weight | estimation |
| number | n | [g] | [g] | [g] | [cm²] | [wt. %] | score |
| Group 1 | 1 | 3230 | 610.99 | 128.63 | 3.871 | 3.98 | 1.20 |
| | 2 | 2890 | 545.18 | 125.68 | 3.814 | 4.35 | 1.32 |
| | 3 | 3015 | 627.96 | 129.06 | 4.113 | 4.28 | 1.36 |
| | 4 | 2720 | 519.97 | 122.27 | 3.690 | 4.50 | 1.36 |
| | 5 | 2810 | 543.65 | 129.64 | 3.791 | 4.61 | 1.35 |
| | 6 | 3095 | 642.44 | 127.69 | 4.037 | 4.13 | 1.30 |
| | | | | | Average value | 4.31 | |
| | | | | | Standard | 0.21 | |
| | | | | | deviation | | |
| Group 2 | 7 | 3005 | 566.48 | 139.33 | 4.391 | 4.64 | 1.46 |
| | 8 | 2530 | 479.95 | 121.66 | 3.622 | 4.81 | 1.43 |
| | 9 | 2830 | 546.99 | 135.20 | 4.537 | 4.78 | 1.60 |
| | 10 | 2885 | 555.87 | 132.02 | 4.386 | 4.58 | 1.52 |
| | 11 | 3285 | 608.10 | 149.73 | 5.127 | 4.56 | 1.56 |
| | 12 | 2645 | 485.66 | 116.00 | 3.798 | 4.39 | 1.44 |
| | 13 | 2470 | 451.35 | 121.85 | 3.924 | 4.93 | 1.59 |
| | | | | | Average value | 4.67 | |
| | | | | | Standard | 0.17 | |
| | | | | | deviation | | |
| Group 3 | 14 | 556 | 110.45 | 16.90 | 0.912 | 3.04 | 1.64 |
| | 15 | 551 | 100.67 | 19.26 | 0.958 | 3.50 | 1.74 |
| | 16 | 545 | 103.89 | 18.38 | 0.977 | 3.37 | 1.79 |
| | 17 | 518 | 105.70 | 17.26 | 0.909 | 3.33 | 1.75 |
| | | | | | Average value | 3.31 | |
| | | | | | Standard | 0.17 | |
| | | | | | deviation | | |

TABLE 10

| | | | | | Cross- | Weight ratio of | Amount of red |
|---|---|---|---|---|---|---|---|
| | | Body | Weight of | Weight of | sectional area | red muscle to | muscle tissue |
| | | Weight | white muscle | red muscle | of red muscle | body weight | estimation |
| | n | [g] | [g] | [g] | [cm²] | [wt. %] | score |
| Group 4 | 18 | 690 | 138.83 | 26.30 | 1.313 | 3.81 | 1.90 |
| | 19 | 590 | 119.25 | 23.16 | 1.151 | 3.93 | 1.95 |
| | 20 | 576 | 112.48 | 22.80 | 1.076 | 3.96 | 1.87 |
| | 21 | 593 | 115.43 | 21.06 | 1.102 | 3.55 | 1.86 |
| | 22 | 684 | 134.40 | 27.95 | 1.267 | 4.09 | 1.85 |
| | 23 | 659 | 125.99 | 24.15 | 1.230 | 3.66 | 1.87 |
| | 24 | 702 | 141.42 | 28.93 | 1.387 | 4.12 | 1.98 |
| | 25 | 537 | 103.00 | 18.13 | 1.064 | 3.38 | 1.98 |
| | 26 | 607 | 123.11 | 18.85 | 1.133 | 3.11 | 1.87 |
| | 27 | 399 | 80.37 | 13.33 | 0.726 | 3.34 | 1.82 |
| | 28 | 481 | 92.08 | 15.86 | 0.937 | 3.30 | 1.95 |
| | 29 | 534 | 99.41 | 19.81 | 0.990 | 3.71 | 1.85 |
| | 30 | 568 | 114.59 | 18.35 | 1.022 | 3.23 | 1.80 |
| | 31 | 625 | 128.80 | 19.94 | 1.202 | 3.19 | 1.92 |
| | | | | | Average value | 3.60 | |
| | | | | | Standard | 0.33 | |
| | | | | | deviation | | |
| Group 5 | 32 | 423 | 83.99 | 14.53 | 0.884 | 3.43 | 2.09 |
| | 33 | 475 | 96.81 | 16.86 | 1.033 | 3.55 | 2.17 |
| | | | | | Average value | 3.49 | |
| | | | | | Standard | 0.06 | |
| | | | | | deviation | | |

### Example 7: Relationship Between Cross-Sectional Area of Pyloric Caeca by Transverse Section Echo Image and Weight of Pyloric Caeca

The pyloric caeca was examined in the same manner as in Examples 1 to 6. Using 10 yellowtails randomly sampled on the sea, a position having a strong correlation was examined from the cross-sectional area of the pyloric caeca determined from the transverse section echo image and the weight of the pyloric caeca actually measured. As a result, it was found that there was a strong correlation between the transverse section echo image of the pelvic fin root and the weight of the pyloric caeca.

The body weight, fork length, and condition factor of these 10 yellowtails determined in the same manner as in Example 1 were as shown in Table 11. From the transverse section echo image of the pelvic fin root of each yellowtail, the cross-sectional area of the pyloric caeca was determined in the same manner as the cross-sectional area of the red muscle of Example 4. Thereafter, the fish body was disassembled, and the pyloric caeca was taken out and weighed using a scale. The cross-sectional area of the pyloric caeca determined from the transverse section echo image and the weight of the pyloric caeca actually measured were as shown in Table 11.

TABLE 11

| | | | | Cross- | | | Pyloric |
|---|---|---|---|---|---|---|---|
| | | | | sectional area | Weight of | Weight ratio of | caeca |
| | Body | Fork | | of pyloric | pyloric | pyloric caeca to | amount |
| | Weight | length | Condition | caeca | caeca | body weight | estimation |
| n | [g] | [cm] | factor | [cm²] | [g] | [%] | score |
| 1 | 5298 | 61.7 | 22.6 | 16.225 | 182.6 | 3.45 | 3.06 |
| 2 | 5240 | 62.2 | 21.8 | 14.636 | 160.3 | 3.06 | 2.79 |
| 3 | 4560 | 58.8 | 22.4 | 12.035 | 128.5 | 2.82 | 2.64 |
| 4 | 5080 | 61.5 | 21.8 | 13.743 | 167.6 | 3.30 | 2.71 |
| 5 | 4730 | 59.6 | 22.3 | 14.075 | 148.3 | 3.14 | 2.98 |
| 6 | 4530 | 59.7 | 21.3 | 12.951 | 150.9 | 3.33 | 2.86 |
| 7 | 4520 | 61.5 | 19.4 | 9.680 | 110.6 | 2.45 | 2.14 |
| 8 | 4510 | 59.8 | 21.1 | 10.767 | 135.1 | 3.00 | 2.39 |
| 9 | 4120 | 57.6 | 21.6 | 7.034 | 82.6 | 2.00 | 1.71 |
| 10 | 5020 | 61.3 | 21.8 | 9.655 | 133.6 | 2.66 | 1.92 |

Using the data shown in Table 11, the product-moment correlation coefficient between the cross-sectional area of the pyloric caeca determined from the transverse section echo image and the weight of the pyloric caeca actually measured was determined. As a result, the product-moment correlation coefficient was 0.94. From this result, it has been found that the weight of the pyloric caeca can also be estimated with high accuracy using the cross-sectional area of the pyloric caeca determined from the echo image.

A pyloric caeca amount estimation score (also referenced as estimated pyloric caeca amount score) was calculated by the following equation. The calculation results were as shown in Table 11.

$$Pyloric caeca amount estimation score = \frac{cross - sectional area of pyloric caeca ( {cm}^{3} )}{body weight ( g )} \times 1000$$

The average value of the weight ratios of the pyloric caeca to the body weights of ten yellowtails shown in Table 11 was 2.92 wt. %, and the standard deviation was 0.42 wt. %. Excluding n=1, which has the maximum pyloric caeca amount estimation score, and n=9, which has the minimum pyloric caeca amount estimation score, the average value of the weight ratios of the pyloric caeca to the body weights of eight yellowtails was 2.97 wt. %, and the standard deviation was 0.29 wt. %. Thus, when the pyloric caeca amount estimation score was used, it was possible to obtain a population with less variation in the weight ratio of the pyloric caeca to the body weight in a non-destructive state.

### INDUSTRIAL APPLICABILITY

According to the present disclosure, an estimation method and an analysis device capable of easily, rapidly, and consistently estimating the amount of marine fish red muscle without exposing the red muscle of the marine fish are provided. An estimation method and an analysis device are provided that are capable of easily estimating the amount of the pyloric caeca of a marine fish without exposing the pyloric caeca and can be used with living fish. There is provided a method for farming a marine fish, the method being capable of efficiently providing a marine fish known to have high market value by reliably and quickly determining the red muscle of the fish to provide marine fish with high market value flexibly, thereby providing products that meet consumer preferences. A method is provided for producing a processed product of a marine fish, the method being capable of efficiently providing a processed product of a marine fish determined to have the red muscle amounts to meet consumer preferences. There is provided a processed product group of a marine fish having a sufficiently reduced variation in the amount of a red muscle and having added value (i.e., a processed marine fish product group with known, consistent red muscle content). There is provided a processed product group of a marine fish having a sufficiently reduced variation in the amount of a pyloric caeca and having added value. An identified product of a marine fish having added value using the methods and materials described herein is also provided.

### REFERENCE SIGNS LIST

10 fish body, 20 non-destructive inspection unit, 22 probe, 24 control unit, 26 first display unit, 30 red muscle estimation unit, 32 calculation unit, 34 second display unit, 50, 50A identified product of marine fish, 52 container, 54, 54A, identification portion, 100 analysis device.

1. A method for estimating an amount of a red muscle, the method comprising the steps of:
acquiring information on a red muscle in a fish body of a marine fish by operating a non-destructive inspection unit; and
estimating an amount of the red muscle included in the fish body based on the acquired information by using a calculation unit.

2. The estimation method according to claim 1, wherein the acquired information includes a cross-sectional area of the red muscle determined from a cross-sectional image of the fish body, and the amount of the red muscle is estimated based on the cross-sectional area in the estimating.

3. The estimation method according to claim 2, further comprising measuring a weight of the fish body, wherein
in the estimating, the weight of the red muscle is estimated based on the weight of the fish body and the cross-sectional area of the red muscle.

4. The estimation method according to claim 1, wherein the non-destructive inspection unit uses at least one selected from the group consisting of ultrasonic inspection, MRI inspection, and CT inspection.

5. The estimation method according to claim 1, wherein the acquired information is acquired from a transverse section image between the fourth vertebra to the twenty-second vertebra of the marine fish or a transverse section image between a second dorsal fin beginning portion and a vicinity of an anus of the marine fish.

6. The estimation method according to claim 1, wherein the acquired information is acquired from a transverse section image between the eleventh vertebra to the seventeenth vertebra or a transverse section image of a vicinity of the anus of the marine fish.

7. A method for producing a processed product of the marine fish, the method comprising a step of processing the marine fish in which the amount of the red muscle has been estimated by the estimation method according to claim 1.

8. A method for farming a marine fish, the method comprising the steps of: determining an estimated value of an amount of a red muscle contained in a marine fish body, and adjusting a farming condition for the marine fish based on the estimated value.

9. The method for farming a marine fish according to claim 8, the method further comprising a step of sorting the marine fish based on the estimated value of the amount of red muscle contained in the marine fish.

10. The method for farming a marine fish according to claim 8, wherein the estimated value is a value estimated based on an image of the red muscle in the fish body acquired by non-destructive inspection.

11. A method for producing a processed product of a marine fish, the method comprising a step of processing the marine fish farmed by the farming method according to claim 8.

12. The method according to claim 1, wherein the method is performed by an analysis device comprising:
the non-destructive inspection unit; and
the calculation unit, wherein the calculation unit is a red muscle estimation unit.

13. The method according to claim 12, wherein the non-destructive inspection unit includes at least one selected from the group consisting of an ultrasonic inspection unit, an MRI inspection unit, and a CT inspection unit.

14. An identified product of a marine fish, comprising:
a fish body or a processed product of a marine fish; and
an identification portion including information indicating that an amount of red muscle contained in the fish body or the processed product, wherein the amount of red muscle has been estimated by non-destructive inspection.

15. The identified product of a marine fish according to claim 14, wherein the identification portion is a machine-readable medium configured to cause a computer system to perform a specific action related to the fish body or the processed product when read by the computer system.

16. A processed product group of a marine fish obtained by processing a first marine fish that is a part of the marine fish sorted in the step of sorting the marine fish in the farming method according to claim 9.
